(19) United States
(12) Patent Application Publication
(10) Pub. No.: US 2016/0346287 A1
(43) Pub. Date: Dec. 1, 2016

(54) METHOD OF TREATING VIRAL INFECTIONS

(71) Applicant: EPIPHANY BIOSCIENCES, INC., San Francisco, CA (US)

(72) Inventors: Fred VOLINSKY, San Francisco, CA (US); Steven Daniel DONG, San Francisco, CA (US)

(21) Appl. No.: 15/234,659

(22) Filed: Aug. 11, 2016

Related U.S. Application Data
(63) Continuation of application No. 14/528,607, filed on Oct. 30, 2014, now abandoned.
Application No. 14/528,607 is a continuation-in-part of application No. PCT/US2013/061190, filed on Sep. 23, 2013.
(60) Provisional application No. 61/703,981, filed on Sep. 21, 2012.

(51) Int. Cl. A61K 31/522; A61K 31/517; A61K 45/06

(52) U.S. Cl. CPC A61K 31/522; A61K 45/06; A61K 31/517

(57) ABSTRACT

The present invention is directed to methods and compositions employing 9-(4-hydroxy-2-(hydroxymethyl)butyl)guanine (“H2G”) or derivatives or analogs thereof for the treatment of viral diseases, as well as for the treatment of other conditions, including, but not limited to, cancer, chronic fatigue syndrome, Alzheimer's disease, multiple sclerosis and Graves' disease. The H2G or derivative or analog thereof can be administered in a pharmaceutical composition and can be administered with an additional antiviral therapeutic agent or another agent for the treatment of other conditions.

### CROSS-REFERENCE TO RELATED APPLICATIONS

This application is a continuation of U.S. patent application Ser. No. 14/528,607 by F. Volinsky and S. D., Dong, filed on Oct. 30, 2014 and entitled “Method of Treating Viral Infections,” which application in turn was a continuation-in-part of PCT Application Serial No. PCT/US2013/061190 by S. Dong and F. Volinsky, designating the United States, filed on Sep. 23, 2013 and entitled “Method of Treating and/or Preventing Shingles and Method of Treating and/or Preventing Zoster Associated Pain,” which in turn claimed the benefit of U.S. Provisional Application Ser. No. 61/703,981 by S. Dong and F. Volinsky, filed Sep. 21, 2012 and entitled “Method of Treating and/or Preventing Shingles and Methods of Treating and/or Preventing Postherpetic Neuralgia.” The contents of both of these applications are incorporated herein by this reference.

### FIELD OF THE INVENTION

This invention is directed to compositions and methods using 9-(4-hydroxy-2-(hydroxymethyl)butyl)guanine (“H2G”) or derivatives or analogs thereof for the treatment of viral diseases.

### BACKGROUND OF THE INVENTION

A number of viral infections, including human herpesviruses, cytomegalovirus, and human immunodeficiency virus (“HIV”) can cause serious diseases. These diseases cause substantial morbidity and may be fatal in patients with compromised immune systems, such as patients undergoing cancer immunotherapy, patients undergoing immunosuppressive treatment to prevent rejection of a tissue transplant, or infants whose immune systems are not completely developed. HIV, itself, causes immunosuppression and leaves patients infected with it susceptible to further infections.

In particular, human herpesvirus 4 (HHV-4) is known as Epstein-Barr virus (EBV). EBV is a member of the γ-herpesvirus family. EBV is an oncogenic virus with tropism for B cells and epithelial cells that establishes persistent and long life in more than 90% of the existing population of the world. Primary infection is usually asymptomatic but may result in infectious mononucleosis. EBV infection can be problematic in certain individuals who develop chronic active EBV as well as in patients who have an immunodeficiency and/or are immunosuppressed, such as transplant receipients and ICU patients. The EBV genome is frequently present in a wide variety of lymphomas, carcinomas and cancers and is believed to contribute to the genesis of these malignancies. In addition, the presence of the EBV genome may also contribute to the development of systemic sclerosis, myalgic encephalomyelitis/chronic fatigue syndrome, systemic lupus erythematosus, idiopathic pulmonary fibrosis, multiple sclerosis or Graves' disease.

Additionally, there is evidence connecting the presence of herpes simplex virus 1 (HSV-1 or HHV-1) infection to the development of Alzheimer's disease.

Accordingly, there is a need for the development of new antiviral treatments for these diseases and indications.

### SUMMARY OF THE INVENTION

The present invention is directed to methods and compositions employing 9-(4-hydroxy-2-(hydroxymethyl)butyl)guanine (“H2G”) or derivatives or analogs thereof for the treatment of viral diseases, as well as for the treatment of other conditions, including, but not limited to, cancer, systemic sclerosis, myalgic encephalomyelitis/chronic fatigue syndrome, Alzheimer's disease, systemic lupus erythematosus, multiple sclerosis and Graves' disease. Derivatives or analogs of H2G include, for example: a monophosphate derivative of H2G, a diphosphate derivative of H2G, or a triphosphate derivative of H2G; a phosphate prodrug analog of H2G; and other compounds, such as the compounds of Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), and Formula (XVII); monophosphate derivatives, diphosphate derivatives, or triphosphate derivatives of these compounds; ethers or esters of H2G or of the compounds of Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), and Formula (XVII); or an alkyl or arylalkyl derivative of a primary hydroxyl group of H2G or of the compounds of of Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), and Formula (XVII).

One aspect of the invention is a method for treatment or prevention of a viral infection comprising the step of administering a therapeutically effective quantity of 9-(4-hydroxy-2-(hydroxymethyl)butyl)guanine (“H2G”) having the structure of Formula (I)

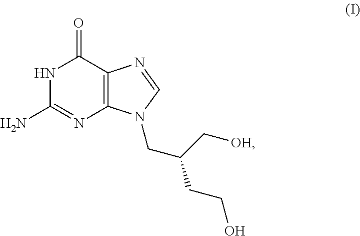 [SMILES: Nc1nc2c(ncn2C[C@H](CO)CCO)c(=O)[nH]1]

or a derivative or analog thereof, for treatment or prevention of the viral infection.

Typically, the wherein the viral infection is selected from the group consisting of a viral infection caused by: (1) human herpesvirus 1 (HHV-1 or HSV-1), a member of the α-herpesvirus subfamily; (2) human herpesvirus 2 (HHV-2 or HSV-2), a member of the α-herpesvirus subfamily; (3) human herpresvirus 3 (HHV-3 or VZV (varicella zoster virus)), a member of the α-herpesvirus subfamily; (4) human herpesvirus 4 (HHV-4 or Epstein-Barr virus), a member of the γ-herpesvirus subfamily; (5) human herpesvirus 5 (HHV-5 or CMV (cytomegalovirus), a member of the β-herpesvirus subfamily; (6) human herpesvirus 6a (HHV-6a), a member of the β-herpesvirus subfamily; (7) human herpesvirus 6b (HHV-6b), a member of the 0-herpesvirus subfamily; (8) human herpesvirus 7 (HHV-7), a member of the β-herpesvirus subfamily; and (9) human herpesvirus 8 (HHV-8 or KSHV (Kaposi's sarcoma associated herpesvirus), a member of the γ-herpesvirus subfamily.

The compound can be administered as the compound itself, but it is generally preferred to administer the compound as a pharmaceutical composition, wherein the pharmaceutical composition comprises: (1) the compound of Formula (I) or the derivative or analog thereof; and (2) at least one pharmaceutically acceptable carrier. Additionally, the scope of the present invention includes any compound subject to activation that, subsequent to activation, creates a triphosphate of H2G or a derivative or analog thereof as described above. Therefore, any compound subject to activation that, when activated, creates a triphosphate of H2G or a derivative or analog thereof as described above, is within the scope of the present invention.

In one alternative, the method can comprise administration of a therapeutically effective quantity of an additional antiviral agent. For example, when the viral infection is is an infection by cytomegalovirus or Epstein-Barr virus and the additional antiviral agent is valganciclovir an infection by HHV-5 (cytomegalovirus), the additional antiviral agent can be letermovir or a derivative or analog of letermovir. When the viral infection is an infection by cytomegalovirus or Epstein-Barr virus, the additional antiviral agent can be valganciclovir. When the viral infection is an infection by cytomegalovirus, the additional antiviral agent can be selected from the group consisting of ganciclovir, cidofovir, and foscarnet. When the viral infection is an infection by Epstein-Barr virus, the additional antiviral agent can be selected from the group consisting of ganciclovir, acyclovir, valaciclovir, cidofovir, adefovir, foscarnet, and romidepsin; for Epstein-Barr virus, alternatively, the additional antiviral agent can be a lytic induction agent. When the viral infection is an infection by herpes simplex virus, the additional antiviral agent can be selected from the group consisting of acyclovir, valaciclovir, famciclovir, and penciclovir. When the viral infection is an infection by human immunodeficiency virus, the additional antiviral agent can be selected from the group consisting of nucleoside reverse transcriptase inhibitors, non-nucleoside reverse transcriptase inhibitors, protease inhibitors, and fusion inhibitors. When the viral infection is an infection by hepatitis C virus, the additional antiviral agent can be selected from the group consisting of sofosbuvir, ribavirin, pegylated interferon-α-2a, pegylated interferon-α-2b, boceprevir, telaprevir, ledipasvir, and simiprevir.

In another alternative, the additional antiviral agent can be at least one one compound selected from the group consisting of valomaciclovir stearate, octadecyloxyethyl-cidofovir, hexadecyloxypropyl-cidofovir, adefovir, amantadine, arbidol, brivudine, darunavir, docosanol, edoxudine, entecavir, fomivirsen, fosfonet, ibacitabine, immunovir, idoxuridine, imiquimod, inosine, loviride, raltegravir, maraviroc, moroxydine, nelfinavir, nexavir, oseltamivir, peramivir, pleconaril, podophyllotoxin, rimantidine, tenofovir, tipranavir, trifluridine, tromantidine, vicriviroc, vidarabine, viramidine, zanamivir, (2-amino-7-[(1,3-dihydroxy-2-propoxy)methyl]purine], (1'S,2′R)-9-[[1′,2′-bis(hydroxymethyl)cycloprop-1′-yl]methyl]guanine (A-5021), cyclopropavir, 2,4-diamino-6-R-[3-hydroxy-2(phosphonomethoxy)propoxy]-pyrimidine, (S)-9-(3-hydroxy-2-phosphonylmethoxypropyl)adenine (S-HPMPA), 3-deaza-9-(3-hydroxy-2-phosphonylmethoxypropyl)adenine (3-deaza-HPMPA), N-(4-chlorobenzyl)-1-methyl-6-(4-morpholinylmethyl)-4-oxo-1,4-dihydro-3-quinolinecarboxamine (PNU-183792), 2-bromo-5,6-dichloro-1-(β-D-ribofuranosyl)benzimidazole (BDCRB), maribavir, 3-hydroxy-2,2-dimethyl-N-[4-{[(5-dimethylamino)-1-naphthyl]-sulfonyl]-amino)phenyl}propamide (BAY 38-4766), N—[N-[4-(2-aminothiazol-4-yl)phenyl]carbamoylmethyl]-N-[1(S)-phenylethyl]pyridine-4-carboxamide (BILS179BS), N-[5-(aminosulfonyl)-4-methyl-1,3-thiazol-2-yl]-N-methyl-2-{4-(2-pyridinyl)phenyl}acetamide (BAY 57-1293), 2H-3-(4-chlorophenyl)-3,4-dihydro-1,4-benzo-thiazine-2-carbonitrile 1,1-dioxide, and 2-chloro-3-pyridin-3-yl-5,6,7,8-tetrahydronindolizine-1-carboxamide (CMV423). In still another alternative, the additional antiviral agent can be an integrase inhibitor, an entry inhibitor, an interferon, or a synergistic enhancer.

Another aspect of the invention is a pharmaceutical composition comprising:

(1) a therapeutically effective quantity of H2G or a derivative or analog thereof; and

(2) a pharmaceutically acceptable carrier.

Derivatives and analogs of H2G are as described above. The pharmaceutical composition can comprise a therapeutically effective quantity of an additional antiviral agent. Additional antiviral agents are as described above.

The pharmaceutical composition can be formulated for oral administration. When the pharmaceutical composition is formulated for oral administration, the pharmaceutical composition can be in a dosage form selected from the group consisting of solutions, suspensions, tablets, dispersible tablets, pills, capsules, troches, granules, bulk powders, sustained release formulations and elixirs.

In another alternative, the pharmaceutical composition is formulated for parenteral administration. When the pharmaceutical composition is formulated for parenteral administration, the pharmaceutical composition can be in a dosage form selected from the group consisting of sterile solutions and suspensions.

In yet other alternatives, the pharmaceutical composition can be formulated for administration via a transdermal patch, for administration via a dry powder inhaler, or can be formulated as an oil-water emulsion.

The pharmaceutical composition can be formulated as a unit-dosage form or as a multiple-dosage form.

The pharmaceutically acceptable carrier can comprise a vehicle selected from the group consisting of water, saline, aqueous dextrose, glycerol, glycols, and ethanol. The pharmaceutically acceptable carrier can comprise a nontoxic auxiliary substance selected from the group of wetting agents, emulsifying agents, solubilizing agents, and pH buffering agents.

In one alternative, the pharmaceutical composition is substantially anhydrous.

The pharmaceutical composition can be in a solid dosage form and include at least one ingredient selected from the group consisting of: a binder; a lubricant; a diluent; a glidant; a disintegrating agent; a coloring agent; a sweetening agent; a flavoring agent; a wetting agent; an emetic coating; and a film coating.

Other alternatives for formulation of the pharmaceutical composition are contemplated. For example, the composition can be formulated in an enteric coating. The composition can include a vehicle selected from the group consisting of aqueous vehicles, nonaqueous vehicles, antimicrobial agents, isotonic agents, buffers, antioxidants, local anesthetics, suspending and dispersing agents, emulsifying agents, sequestering agents, and chelating agents. The composition can be formulated as a sterile lyophilized powder, as a topical mixture, as an aerosol for topical application, for topical application to the skin or mucous membranes, or for rectal administration.

In yet another alternative, the pharmaceutical composition is formulated to target the therapeutically effective quantity of H2G or the derivative or analog thereof, or the therapeutically effective quantity of an additional antiviral agent, to a particular organ, tissue, receptor, or other area of the body of a subject to be treated.

Another aspect of the invention is an article of manufacture comprising:

(1) a therapeutically effective quantity of H2G or a derivative or analog of H2G as described above;

(2) packaging material to package the therapeutically effective quantity of H2G or a derivative or analog of H2G; and

(3) a label indicating that the H2G or the derivative or analog thereof is useful for treating a viral infection and providing instructions for its use.

Yet another aspect of the invention is an article of manufacture comprising:

(a) a therapeutically effective quantity of H2G or a derivative or analog of H2G as described above;

(b) an additional antiviral agent;

(c) packaging material to package the therapeutically effective quantity of H2G or a derivative or analog of H2G and the additional antiviral agent; and

(d) a label indicating that the H2G or the derivative or analog thereof and the additional antiviral agent are useful for treating a viral infection and providing instructions for its use.

Yet another aspect of the invention is a method for the treatment of a disease or condition selected from the group consisting of systemic sclerosis, myalgic encephalomyelitis/chronic fatigue syndrome, Alzheimer's disease, systemic lupus erythematosus, multiple sclerosis and Graves' disease comprising administering a therapeutically effective quantity of H2G or a derivative or analog thereof for treatment of the disease or condition. The H2G or the derivative or analog thereor can be administered in a pharmaceutical composition, and can be administered with another agent to treat one of these diseases or conditions.

Yet another aspect of the invention is a method for the treatment of cancer comprising administering a therapeutically effective quantity of H2G or a derivative or analog thereof for treatment of the cancer. Typically, the cancer is selected from the group consisting of gastric carcinoma, lymphoma, Hodgkin's disease, nasopharygeal carcinoma, breast cancer, lung cancer, colon cancer, and prostate cancer. The H2G or derivative or analog can be administered as a pharmaceutical composition. The method can further comprise the administration of an additional anti-neoplastic agent.

### DETAILED DESCRIPTION OF THE INVENTION

The present invention is directed to methods and compositions employing 9-(4-hydroxy-2-(hydroxymethyl)butyl)guanine (“H2G”) or derivatives or analogs thereof for the treatment of viral diseases.

H2G, also known as omaciclovir and 2HM-Hbg, has the structure shown in Formula (I)

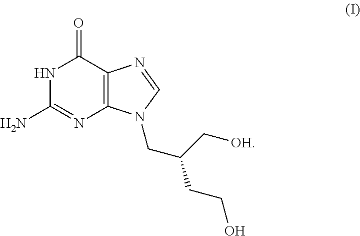 [SMILES: Nc1nc2c(ncn2C[C@H](CO)CCO)c(=O)[nH]1]

H2G is phosphorylated in vivo by viral thymidine kinase, typically to the triphosphate form, which then acts as an antimetabolite to block viral replication. The activity of H2G has been described in K. Yao et al., “Effect of (r)-9-[4-Hydroxy-2-(hydroxymethyl)butyl]guanine (H2G) and AZT-lipid-PFA on Human Herpesvirus-6B Infected Cells,” J. Clin. Virol. 46: 10-14 (2009), incorporated herein by this reference.

The following definitions are used in this application and apply unless specifically stated to the contrary:

“Compounds” refers to compounds encompassed by structural formulae disclosed herein and includes any specific compounds within these formulae whose structure is disclosed herein. Compounds may be identified either by their chemical structure and/or chemical name. When the chemical structure and chemical name conflict, the chemical structure is determinative of the identity of the compound. Specific chemical structures are shown where appropriate and indicated by a specific formula designation with a Roman numeral, such as “Formula (I).”

The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures unless some alternatives are excluded. Enantiomeric and stereoisomeric mixtures can be resolved into their component enantiomers or stereoisomers using separation techniques known in the art. Alternatively chiral synthesis techniques well known in the art can be used to synthesize specific enantiomers or diastereomers. The compounds may also exist in several tautomeric forms including the enol form, the keto form and mixtures thereof. Other forms of tautomerism, such as imine-enamine tautomerism, are also known in the art. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to, ²H, ³H, ¹³C, ¹⁴N, ¹⁵N, ¹⁷O, ¹⁸O, or other isotopes. Compounds may exist in unsolvated forms as well as solvated forms, including hydrated forms and as N-oxides. In general, compounds may be hydrated, solvated or N-oxides. Certain compounds may exist in multiple crystalline or amorphous forms.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl, prop-2-yn-1-yl, or other groups known in the art; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, and other groups known in the art. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

The term “alkanyl,” by itself or as part of another substituent, refers to a saturated branched, straight-chain or cyclic alkyl radical derived by the removal of one hydrogen atom from a single carbon atom of a parent alkane. Typical alkanyl groups include, but are not limited to, methanyl; ethanyl; propanyls such as propan-1-yl, propan-2-yl (isopropyl), cyclopropan-1-yl, and other groups known in the art; butanyls such as butan-1-yl, butan-2-yl (sec-butyl), 2-methyl-propan-1-yl (isobutyl), 2-methyl-propan-2-yl (t-butyl), cyclobutan-1-yl, and other groups known in the art.

The term “alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s), leading to geometrical isomerism, unless a specific. Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, and other groups known in the art.

The term “alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, and other groups known in the art; butynyls such as but-1-yn-1-yl, but-1-yn-3-yl, but-3-yn-1-yl, and other groups known in the art.

The term “alkoxy,” by itself or as part of another substituent, refers to a radical of the formula —O—R^{a}, wherein R^{a} refers to alkyl as defined herein.

The term “alkyalkoxy,” by itself or as part of another substituent, refers to a radical of the form —R^{a}—O—R^{b}, wherein R^{a} and R^{b} each refer to alkyl as defined herein. The groups R^{a} and R^{b} may be the same or different.

The term “aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and other groups known in the art. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 15 carbon atoms (C₆-C₁₀ aryl).

The term “arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In some embodiments, an arylalkyl group is (C₆-C₃₀) arylalkyl, e.g., the alkanyl, alkenyl or alkynyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkanyl, alkenyl or alkynyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkanyl, alkenyl or alkynyl moiety of the arylalkyl group is (C₁-C₅) alkyl and the aryl moiety is (C₆-C₁₀) aryl. Other combinations of aryl and alkyl groups are known in the art.

The term “salts,” as used herein refers to salts of the compounds disclosed herein such as, for example, salts of organic acids, especially carboxylic acids, including but not limited to acetate, trifluoroacetate, lactate, gluconate, citrate, tartrate, maleate, malate, pantothenate, isethionate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulfonic acids such as methanesulfonate, ethanesulphonate, 2-hydroxyethane sulfonate, camphorsulfonate, 2-napthalenesulfonate, benzenesulfonate, p-chlorobenzenesulfonate and p-toluenesulfonate; and inorganic acids such as hydrochloride, hydrobromide, hydroiodide, sulfate, bisulfate, hemisulfate, thiocyanate, persulfate, phosphoric and sulfonic acids. Hydrochloric acid salts are convenient in many applications and are generally well-tolerated pharmacologically.

The term “vehicle” as used herein refers to a diluent, adjuvant, excipient or carrier with which an antiviral agent or another therapeutic agent is administered.

The terms “subject,” “individual,” or “patient” as used herein are used interchangeably herein and refer to a vertebrate, preferably, a mammal. Mammals include, but are not limited to, murines, rodents, simians, humans, farm animals, sport animals and pets. Unless specifically stated to be so limited, methods and compositions according to the present invention are not limited to treatment of or use in humans.

The terms “preventing,” “prevention,” or similar terminology, as used herein, refer to a reduction in risk of acquiring a disease or disorder (i.e., causing at least one of the clinical symptoms of the disease not to develop in a subject that may be exposed to or predisposed to the disease but does not yet experience or display symptoms of the disease).

The terms “treating,” “treatment,” or similar terminology, refer, in some embodiments, to ameliorating the disease or disorder (i.e., arresting or reducing the development of the disease or at least one of the clinical symptoms thereof). In other embodiments “treating” or “treatment” refers to ameliorating at least one physical parameter, which may not be discernible by the subject. In yet other embodiments, “treating” or “treatment” refers to inhibiting the disease or disorder, either physically (e.g., stabilization of a discernible symptom), physiologically (e.g., stabilization of a physical parameter) or both. This can be demonstrated by, for example, but not by way of limitation, reduction in viral load, reduction of fever, reduction of pain, reduction of malaise, reduction of tissue destruction, or improvement in other clinical parameters. In yet other embodiments, “treating” or “treatment” refers to delaying the onset of the disease or disorder. Use of the terms “treating,” “treatment,” or similar terminology is not to be taken as stating or implying a cure for any disease or condition.

The term “therapeutically effective amount,” as used herein means the amount of an antiviral agent or other compound that, when administered to a subject for treating a disease, is sufficient to effect such treatment for the disease. The “therapeutically effective amount” will vary depending on the antiviral agent or other compound being administered, the disease and its severity, the age and weight of the subject to be treated, other therapeutic agents being administered to the subject, and pharmacokinetic factors such as liver and kidney function.

Compounds

Compounds for use in methods and compositions according to the present invention include 9-(4-hydroxy-2-(hydroxymethyl)butyl)guanine (“H2G”), which has the structure shown in Formula (I)

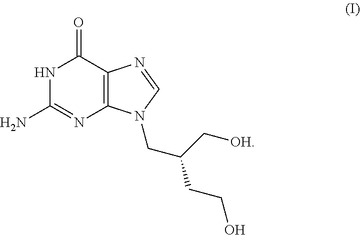 [SMILES: Nc1nc2c(ncn2C[C@H](CO)CCO)c(=O)[nH]1]

Additionally, monophosphate, diphosphate, and triphosphate derivatives of H2G are within the scope of the present invention and can be used in methods and compositions according to the present invention.

Also within the scope of the present invention are phosphate prodrug analogs of H2G. These phosphate prodrug analogs can be used in methods and compositions according to the present invention.

Prodrugs for guanine derivatives and analogs are described, for example, in United States Patent Application Publication No. 2003/0186924 by Zhou et al., incorporated herein by this reference, which discloses prodrugs incorporating glyceryl groups and trifunctional linkers and which may contain fluoro groups; the prodrugs can include fatty acid moieties that can also be used as linkers. Further prodrugs are described in United States Patent Application Publication No. 2005/0250795 by Leanna et al., incorporated herein by this reference, which discloses aliphatic ester prodrugs, such as di-pivaloyl, di-valeroyl, mono-valeroyl, mono-oleoyl and mono-stearoyl esters. Still further prodrugs are described in United States Patent Application Publication No. 2006/0122383 by Zhou et al., incorporated by this reference, which discloses double prodrugs that can include trifunctional linker groups. Still further prodrugs are disclosed in United States Patent Application Publication No. 2010/0227833 by Yin et al., incorporated herein by this reference, which discloses esters and amides as prodrugs. Still further prodrugs are disclosed in United States Patent Application No. 2011/0065738 by Tulshian et al., incorporated herein by this reference, which discloses: (i) esters formed from acid groups such as by the replacement of the hydrogen of the free acid with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, γ-butyrolacton-4-yl, di-N,N—(C₁-C₂)alkylamino(C₂-C₃)alkyl (such as β-dimethylaminoethyl), carbamoyl(C₁-C₂)alkyl, N,N-di (C₁-C₂)alkylcarbamoyl(C₁-C₂)alkyl and piperidino-, pyrrolidino- or morpholino(C₂-C₃)alkyl, and the like; (ii) prodrugs formed by replacement of the hydrogen of an alcohol moiety with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N—(C₁-C₆)alkoxycarbonylaminomethyl, succinoyl, (C₁-C₆)alkanoyl, α-amino(C₁-C₄)alkyl, α-amino(C₁-C₄)alkylene-aryl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like; or (iii) prodrugs formed by replacement of a hydrogen atom in an amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

Other prodrugs are known in the art. The use of prodrug systems is described in T. Järvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796, incorporated herein by this reference.

Also within the scope of the present invention is an analog of H2G in which the purine moiety is 2-amino-9-purine. This analog has the structure of Formula (II)

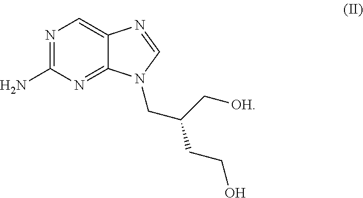 [SMILES: Nc1ncc2ncn(C[C@H](CO)CCO)c2n1]

Further, within the scope of the present invention are monophosphate, diphosphate, and triphosphate derivatives of the analog of H2G of Formula (II).

Still other analogs of H2G are within the scope of the invention, including, but not limited to, analogs of Formula (III)

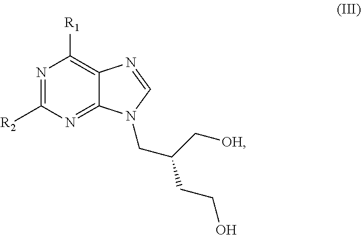

wherein:
(1) R₁ is hydrogen, hydroxy, mercapto, or amino; and
(2) R₂ is hydrogen, hydroxy, fluoro, chloro, or amino;
   and monophosphate derivatives, diphosphate derivatives, or triphosphate derivatives of analogs of Formula (III).

Still other analogs of H2G are within the scope of the invention, including, but not limited to, analogs of Formula (IV):

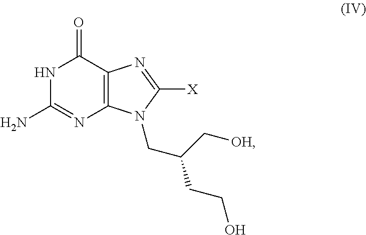

wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (IV).

Still other analogs of H2G are within the scope of the invention, including, but not limited to, analogs of Formula (V):

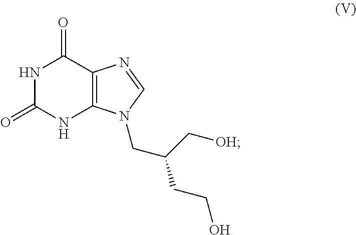 [SMILES: O=c1[nH]c(=O)c2ncn(C[C@H](CO)CCO)c2[nH]1]

and monophosphate derivatives, diphosphate derivatives, or triphosphate derivatives of analogs of Formula (V).

Still other analogs of H2G are within the scope of the present invention, including, but not limited to, compounds of Formula (VI):

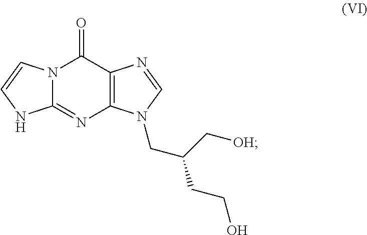 [SMILES: O=c1c2ncn(C[C@H](CO)CCO)c2nc2[nH]ccn12]

and monophosphate derivatives, diphosphate derivatives, or triphosphate derivatives of analogs of Formula (VI).

Still other analogs of H2G are within the scope of the present invention, including, but not limited to, compounds of Formula (VII):

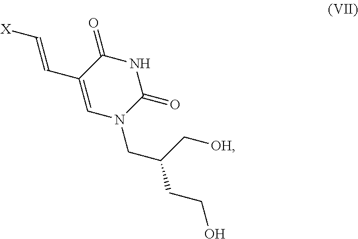

wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (VII).

Still other analogs of H2G are within the scope of the present invention, including, but not limited to, compounds of Formula (VIII):

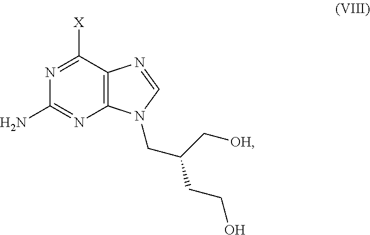

wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (VIII).

Still other analogs of H2G are within the scope of the present invention, including, but not limited to, compounds of Formula (IX):

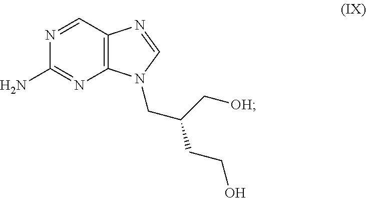 [SMILES: Nc1ncc2ncn(C[C@H](CO)CCO)c2n1]

and monophosphate derivatives, diphosphate derivatives, or triphosphate derivatives of analogs of Formula (IX).

Still other analogs of H2G are within the scope of the present invention, including, but not limited to, compounds of Formula (X):

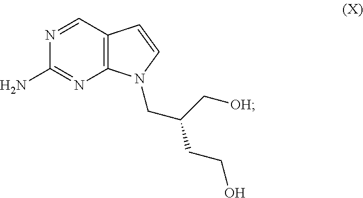 [SMILES: Nc1ncc2ccn(C[C@H](CO)CCO)c2n1]

and monophosphate derivatives, diphosphate derivatives, or triphosphate derivatives of analogs of Formula (X).

Still other analogs of H2G are within the scope of the present invention, including, but not limited to, compounds of Formula (XI):

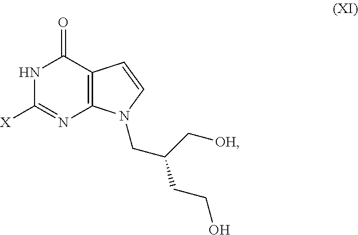

wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (XI).

Still other analogs of H2G are within the scope of the present invention, including, but not limited to, compounds of Formula (XII):

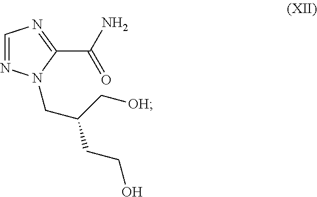 [SMILES: NC(=O)c1ncnn1C[C@H](CO)CCO]

and monophosphate derivatives, diphosphate derivatives, or triphosphate derivatives of analogs of Formula (XII).

Still other analogs of H2G are within the scope of the present invention, including, but not limited to, compounds of Formula (XIII):

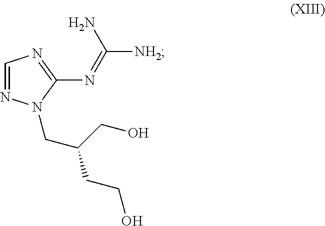 [SMILES: NC(N)=Nc1ncnn1C[C@H](CO)CCO]

and monophosphate derivatives, diphosphate derivatives, or triphosphate derivatives of analogs of Formula (XIII).

Still other analogs of H2G are within the scope of the present invention, including, but not limited to, compounds of Formula (XIV):

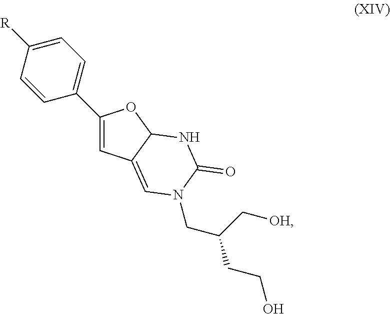

wherein R is selected from the group consisting of —(CH₂)ₙ—CH₃ wherein n is an integer from 0 to 11 and -(Phenyl)-p-(CH₂)ₙ—CH₃ wherein n is an integer from 1 to 10;
and monophosphate derivatives, diphosphate derivatives, or triphosphate derivatives of analogs of Formula (XIV).

Still other analogs of H2G are within the scope of the present invention, including, but not limited to, compounds of Formula (XV):

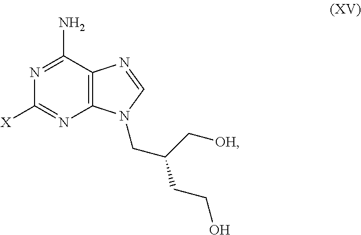

wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (XV).

Still other analogs of H2G are within the scope of the present invention, including, but not limited to, compounds of Formula (XVI):

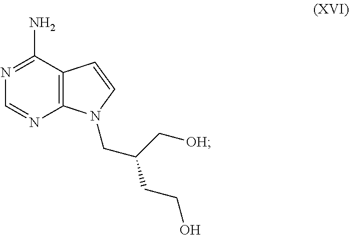 [SMILES: Nc1ncnc2c1ccn2C[C@H](CO)CCO]

and monophosphate derivatives, diphosphate derivatives, or triphosphate derivatives of analogs of Formula (XVI).

Still other analogs of H2G are within the scope of the present invention, including, but not limited to, compounds of Formula (XVII):

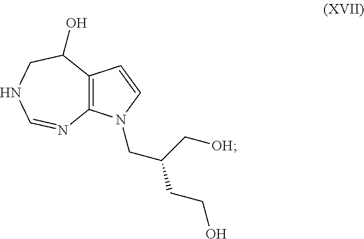 [SMILES: OCC[C@@H](CO)Cn1ccc2c1N=CNCC2O]

and monophosphate derivatives, diphosphate derivatives, or triphosphate derivatives of analogs of Formula (XVII).

Additionally, esters and ethers of the above compounds are also useful antiviral agents. Examples of esters are phosphate esters, carboxylic esters, carbonate esters, carbamate esters or sulfonic esters. The acid part of the esters may have alkyl, aryl or arylalkyl chains, where the aryl functionalities are optionally substituted for example by alkoxy, amino, nitrile, alkyl or sulfonamido groups or by one or more halogen atoms. These esters and ethers include, but are not limited to, esters and ethers of compounds of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), and Formula (XVII).

Other types of derivatives of the above compounds which may be useful as antiviral agents include alkyl or arylalkyl derivatives of the primary hydroxyl group(s). The arylalkyl ether derivatives may be for example benzyl or triphenylmethyl and the aryl moiety may be optionally substituted. These alkyl or aralkyl derivatives include, but are not limited to, esters and ethers of compounds of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), and Formula (XVII), where a suitable primary hydroxyl group exists.

Therapeutic Use of Compounds

One aspect of the present invention is the use of the compounds described above to treat or prevent a viral infection. Typically, a method according to the present invention for treatment or prevention of a viral infection comprises the step of administering a therapeutically effective quantity of a compound according to the present invention to a subject suffering from a viral disease treatable by the compound, or at risk of contracting a viral disease treatable by the compound.

Viral diseases treatable or preventable by compounds according to the present invention include, but are not limited to diseases caused by: (1) human herpesvirus 1 (HHV-1 or HSV-1), a member of the α-herpesvirus subfamily; (2) human herpesvirus 2 (HHV-2 or HSV-2), a member of the α-herpesvirus subfamily; (3) human herpresvirus 3 (HHV-3 or VZV (varicella zoster virus)), a member of the α-herpesvirus subfamily; (4) human herpesvirus 4 (HHV-4 or Epstein-Barr virus), a member of the γ-herpesvirus subfamily; (5) human herpesvirus 5 (HHV-5 or CMV (cytomegalovirus)), a member of the β-herpesvirus subfamily; (6) human herpesvirus 6a (HHV-6a), a member of the β-herpesvirus subfamily; (7) human herpesvirus 6b (HHV-6b), a member of the β-herpesvirus subfamily; (8) human herpesvirus 7 (HHV-7), a member of the (3-herpesvirus subfamily; and (9) human herpesvirus 8 (HHV-8 or KSHV (Kaposi's sarcoma associated herpesvirus), a member of the γ-herpesvirus subfamily. The designation “HSV” (herpes simplex virus) refers to either human herpesvirus 1 or human herpesvirus 2.

A particularly significant use of compounds and pharmaceutical compositions according to the present invention is to decrease time required to be spent on ventilators for patients in an intensive care unit (ICU) that are infected with Epstein-Barr virus (HHV-4). Further details about the use of compounds and pharmaceutical compositions according to the present invention are provided below.

Viral diseases treatable by compounds according to the present invention also include disease caused by hepatitis C virus (HCV). Viral diseases treatable by compounds according to the present invention also include diseases caused by human immunodeficiency virus (HIV) and animal herpesviruses including simian varicella virus (SVV).

The compound according to the present invention can be administered as the compound itself. However, as detailed further below, the compound according to the present invention is typically administered in the form of a pharmaceutical composition. Additionally, the scope of the present invention includes any compound subject to activation that, subsequent to activation, creates a triphosphate of H2G or a derivative or analog thereof as described above. Therefore, any compound subject to activation that, when activated, creates a triphosphate of H2G or a derivative or analog thereof as described above, is within the scope of the present invention for administration to treat viral diseases as described above.

In one alternative, the compound according to the present invention is administered as a single therapeutic agent.

In another alternative, the compound according to the present invention can be administered together with another therapeutic agent for prevention or treatment of a disease caused by a virus as described above. The additional therapeutic agent will depend on the particular viral disease to be treated or prevented.

When the virus is HHV-5 (cytomegalovirus), a suitable additional therapeutic agent is letermovir or a derivative or analog of letermovir. Preferably, the additional therapeutic agent is letermovir.

Letermovir has the systematic (IUPAC) name of {(4S)-8-fluoro-2-[4-(3-methoxyphenyl)-1-piperazinyl]-3-[2-methoxy-5-(trifluoromethyl)phenyl]-3,4-dihydro-4-quinazolinyl}acetic acid and the structure shown in Formula (XVIII):

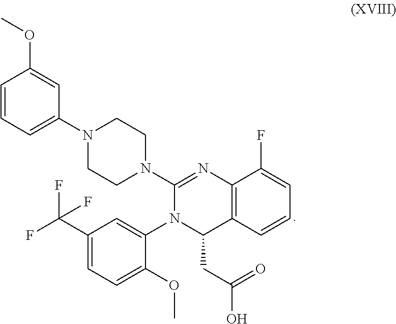 [SMILES: COc1cccc(N2CCN(C3=Nc4c(F)cccc4[C@H](CC(=O)O)N3c3cc(C(F)(F)F)ccc3OC)CC2)c1]

The synthesis and activity of letermovir is disclosed in U.S. Pat. No. 7,196,086 to Wunberg et al., incorporated herein by this reference.

Letermovir, and derivatives and analogs of letermovir as described below, shows antiviral activity on representatives of the Herpesviridae, especially on cytomegaloviruses such as human cytomegalovirus.

Letermovir can be administered by a number of conventional routes, including, but not limited to, oral, parenteral, pulmonary, nasal, sublingual, lingual, buccal, rectal, dermal, transdermal, conjunctival, or otic routes, or by implant or stent. Parenteral administration can take place with avoidance of an adsorption step, such as by intravenous, intra-arterial, intracardial, intraspinal, or intralumbar routes, or with inclusion of absorption, such as by intramuscular, subcutaneous, intracutaneous, percutaneous, or intraperitoneal routes.

Suitable dosage forms for the administration of letermovir include, for oral administration, dosage forms that deliver the compounds according to the invention rapidly and/or in modified form and which comprise the compounds according to the invention in crystalline and/or amorphous and/or dissolved form, such as, for example, tablets (uncoated or coated tablets, for example tablets provided with enteric coatings or coatings which dissolve slowly or are insoluble and which control the release of the compound according to the invention), tablets which disintegrate rapidly in the oral cavity and/or films/wafers, films/lyophilizates, capsules (for example hard or soft gelatin capsules), sugar-coated tablets, granules, pellets, powders, emulsions, suspensions, aerosols or solutions. For parenteral administration, suitable dosage forms include preparations for injection and infusion in the form of solutions, suspensions, emulsions, lyophilizates or sterile powders. Suitable dosage forms for other administration routes include pharmaceutical forms for inhalation (inter alia powder inhalers, nebulizers), nasal drops/solutions/sprays; tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems, milk, pastes, foams, dusting powders, implants or stents.

Typically, suitable dosages for intravenous administration of letermovir are from about 0.001 mg/kg bodyweight/day to about 10 mg/kg bodyweight/day, preferably about 0.01 mg/kg bodyweight/day to about 5 mg/kg bodyweight/day to achieve effective results; suitable dosages for oral administration are from about 0.01 to about 25 mg/kg bodyweight/day, preferably from about 0.1 mg/kg bodyweight/day to about 10 mg/kg bodyweight/day. However, these dosages can be adjusted depending on a number of factors including the severity of the condition to be treated, the age, weight, and sex of the patient to be treated, other medications being administered to the patient to be treated, such as immunosuppressive agents, and pharmacokinetic factors such as liver and kidney function and will ultimately be at the discretion of the attendant physician.

U.S. Pat. No. 7,196,086 to Wunberg et al., incorporated herein by this reference, also discloses derivatives and analogs of letermovir.

One class of derivatives or analogs of letermovir are compounds of Formula (XIX):

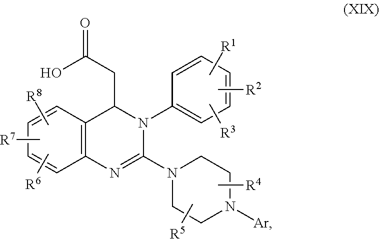

wherein:
(1) Ar represents aryl which may be substituted by 1 to 3 substituents, where the substituents are selected independently of one another from the group consisting of alkyl, alkoxy, formyl, carboxyl, alkylcarbonyl, alkoxycarbonyl, trifluoromethyl, halogen, cyano, hydroxyl, amino, alkylamino, aminocarbonyl and nitro, where alkyl may be substituted by 1 to 3 substituents, where the substituents are selected independently of one another from the group consisting of halogen, amino, alkylamino, hydroxyl and aryl, or two of the substituents on the aryl radical together with the carbon atoms to which they are attached form a 1,3-dioxolane, a cyclopentane ring or a cyclohexane ring, and any third substituent present is selected independently from the group mentioned;
(2) R¹ represents hydrogen, amino, alkyl, alkoxy, alkylamino, alkylthio, cyano, halogen, nitro or trifluoromethyl;
(3) R² represents hydrogen, alkyl, alkoxy, alkylthio, cyano, halogen, nitro or trifluoromethyl;
(4) R³ represents amino, alkyl, alkoxy, alkylamino, alkylthio, cyano, halogen, nitro, trifluoromethyl, alkylsulfonyl or alkylaminosulfonyl; or
(5) one of the radicals R¹, R² and R.sup.3 represents hydrogen, alkyl, alkoxy, cyano, halogen, nitro or trifluoromethyl and the other two together with the carbon atoms to which they are attached form a 1,3-dioxolane moiety, a cyclopentane ring or a cyclohexane ring;
(6) R⁴ represents hydrogen or alkyl;
(7) R⁵ represents hydrogen or alkyl; or
(8) R⁴ and R⁵ are attached to carbon atoms directly opposing each other in the piperazine ring and form a methylene bridge which is optionally substituted by 1 or 2 methyl groups;
(9) R⁶ represents alkyl, alkoxy, alkylthio, formyl, carboxyl, aminocarbonyl, alkylcarbonyl, alkoxycarbonyl, trifluoromethyl, halogen, cyano, hydroxyl or nitro;
(10) R⁷ represents hydrogen, alkyl, alkoxy, alkylthio, formyl, carboxyl, alkylcarbonyl, alkoxycarbonyl, trifluoromethyl, halogen, cyano, hydroxyl or nitro; and
(11) R⁸ represents hydrogen, alkyl, alkoxy, alkylthio, formyl, carboxyl, alkylcarbonyl, alkoxycarbonyl, trifluoromethyl, halogen, cyano, hydroxyl or nitro; and the salts of these compounds, solvates of these compounds, and solvates of salts of these compounds.

For these compounds, physiologically acceptable salts include, but are not limited to, acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid. Physiologically acceptable salts also include, but are not limited to, salts of customary bases, such as, by way of example and preferably, alkali metal salts (for example sodium and potassium salts), alkaline earth metal salts (for example calcium and magnesium salts) and ammonium salts derived from ammonia or organic amines having 1 to 16 carbon atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

For substituents in compounds of Formula (XIX), the substituents are defined as follows:
(1) alkyl per se and “alk” or “alkyl” in alkoxy, alkylamino, alkylcarbonyl, alkylsulfonyl, alkylaminosulfonyl and alkoxycarbonyl or analogous groups are a straight-chain or branched alkyl radical having generally 1 to 6, preferably 1 to 4, particularly preferably 1 to 3, carbon atoms, by way of example and preferably methyl, ethyl, n-propyl, isopropyl, t-butyl, n-pentyl and n-hexyl;
(2) alkoxy is, by way of example and preferably, methoxy, ethoxy, n-propoxy, isopropoxy, t-butoxy, n-pentoxy and n-hexoxy;
(3) alkylamino is an alkylamino radical having one or two alkyl substituents (chosen independently of one another), by way of example and preferably methylamino, ethylamino, n-propylamino, isopropylamino, t-butylamino, n-pentylamino, n-hexylamino, N,N-dimethylamino, N,N-diethylamino, N-ethyl-N-methylamino, N-methyl-N-n-propylamino, N-isopropyl-N-n-propyl-amino, N-t-butyl-N-methylamino, N-ethyl-N-n-pentylamino and N-n-hexyl-N-methylamino; C₁-C₃-alkylamino is, for example, a monoalkylamino radical having 1 to 3 carbon atoms or a dialkylamino radical having in each case 1 to 3 carbon atoms per alkyl substituent;
(4) alkylsulfonyl is, by way of example and preferably, methylsulfonyl, ethylsulfonyl, n-propyl-sulfonyl, isopropylsulfonyl, t-butylsulfonyl, n-pentylsulfonyl and n-hexylsulfonyl;
(5) alkylaminosulfonyl is an alkylaminosulfonyl radical having one or two alkyl substituents (chosen independently of one another), by way of example and preferably methylaminosulfonyl, ethylaminosulfonyl, n-propylaminosulfonyl, isopropylaminosulfonyl, t-butylaminosulfonyl, n-pentylaminosulfonyl, n-hexyl-aminosulfonyl, N—N-dimethylaminosulfonyl, N—N-diethyl-aminosulfonyl, N-ethyl-N-methylaminosulfonyl, N-methyl-N-n-propylaminosulfonyl, N-isopropyl-N-n-propylaminosulfonyl, N-t-butyl-N-methylaminosulfonyl, N-ethyl-N-n-pentyl-aminosulfonyl and N-n-hexyl-N-methylaminosulfonyl; C₁-C₃-alkylaminosulfonyl is, for example, a monoalkylaminosulfonyl radical having 1 to 3 carbon atoms or a dialkylaminosulfonyl radical having in each case 1 to 3 carbon atoms per alkyl substituent;
(6) alkylcarbonyl is, by way of example and preferably, acetyl and propanoyl;
(7) alkoxycarbonyl is, by way of example and preferably, methoxycarbonyl, ethoxycarbonyl, n-propoxycarbonyl, isopropoxycarbonyl, t-butoxycarbonyl, n-pentoxycarbonyl and n-hexoxycarbonyl;
(8) aryl is a mono- to tricyclic aromatic carbocyclic radical having generally 6 to 14 carbon atoms; by way of example and preferably phenyl, naphthyl and phenanthrenyl; and
(9) halogen is fluorine, chlorine, bromine and iodine, preferably fluorine and chlorine.

Compounds of Formula (XIX) are preferred in which Ar represents phenyl which may be substituted by 1 to 3 substituents, where the substituents are selected independently of one another from the group consisting of C₁-C₆-alkyl, C₁-C₆-alkoxy, carboxyl, C₁-C₆-alkylcarbonyl, C₁-C₆-alkoxycarbonyl, trifluoromethyl, fluorine, chlorine, bromine, cyano, hydroxyl, amino, C₁-C₆-alkylamino and nitro, or two of the substituents on the phenyl radical together with the carbon atoms to which they are attached form a 1,3-dioxolane and any third substituent present is selected independently from the group mentioned; R¹ represents hydrogen, C₁-C₃-alkyl, C₁-C₃-alkoxy, C₁-C₃-alkylthio, fluorine or chlorine, R² represents hydrogen, C₁-C₃-alkyl, C₁-C₃-alkoxy, C₁-C₃-alkylthio, fluorine or chlorine; R³ represents C₁-C₄-alkyl, cyano, fluorine, chlorine, nitro, trifluoromethyl or C₁-C₃-alkylsulfonyl, or one of the radicals R¹, R² and R³ represents hydrogen, C₁-C₃-alkyl, C₁-C₃-alkoxy, cyano, halogen, nitro or trifluoromethyl and the other two together with the carbon atoms to which they are attached form a cyclopentane ring or a cyclohexane ring; R⁴ represents hydrogen or methyl; R⁵ represents hydrogen; R⁶ represents C₁-C₃-alkyl, C₁-C₃-alkoxy, carboxyl, aminocarbonyl, trifluoromethyl, fluorine, chlorine, cyano, hydroxyl or nitro, R⁷ represents hydrogen, C₁-C₃-alkyl, C₁-C₃-alkoxy, fluorine, chlorine, cyano or hydroxyl; and R⁸ represents hydrogen, C₁-C₃-alkyl, C₁-C₃-alkoxy, fluorine, chlorine, cyano or hydroxyl.

Among these, particular preference is given to those compounds of Formula (XIX), in which Ar represents phenyl which may be substituted by 1 or 2 substituents, where the substituents are selected independently of one another from the group consisting of methyl, methoxy, fluorine and chlorine; R¹ represents hydrogen, methyl, methoxy, methylthio, fluorine or chlorine; R² represents hydrogen; R³ represents methyl, isopropyl, t-butyl, cyano, fluorine, chlorine, nitro or trifluoromethyl; R⁴ represents hydrogen; R⁵ represents hydrogen; R⁶ represents aminocarbonyl, fluorine, chlorine, cyano or hydroxyl; R⁷ represents hydrogen; and R⁸ represents hydrogen, fluorine or chlorine.

Additional compounds of Formula (XIX) are preferred in which R¹ represents hydrogen, methyl, methoxy or fluorine.

Additional compounds of Formula (XIX) are also preferred in which R¹ is attached to the phenyl ring via the position ortho to the point of attachment of the phenyl ring. For these compounds of Formula (XIX) as used herein in compositions and methods according to the present invention, the point of attachment of the phenyl ring substituted by radicals R¹, R², and R³ is to be understood as meaning the carbon atom of the phenyl ring which, according to Formula (XIX), is attached to one of the two nitrogen atoms of the dihydroquinazoline. Among these compounds are compounds of Formula (XIX) in which R¹ represents methoxy and R¹ is attached to the phenyl ring via the position ortho to the point of attachment of the phenyl ring. Also among these compounds are compounds of Formula (XIX) in which R² represents hydrogen. Also among these compounds are compounds of Formula (V) in which R³ represents trifluoromethyl, chlorine, methyl, isopropyl or t-butyl.

Additional compounds of Formula (XIX) are also preferred in which R¹ is attached to the phenyl ring via the position ortho to the point of attachment of the phenyl ring and R³ is attached to the phenyl ring via the position meta to the point of attachment of the phenyl ring, which position is opposite to that of R¹. In these compounds, R³ typically represents trifluoromethyl, chlorine, or methyl.

Additional compounds of Formula (XIX) that are preferred include compounds in which R⁴ and R⁵ represent hydrogen.

Additional compounds of Formula (XIX) that are preferred include compounds in which R⁶ represents fluorine. In such compounds, it is particularly preferred that the fluorine is attached to the aromatic six-membered ring of the dihydroquinazoline moiety as shown in Formula (XIX(a)):

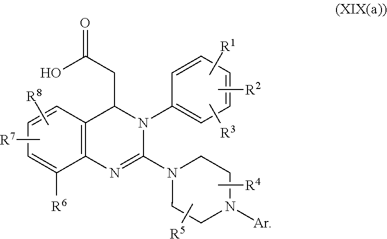

Additional compounds of Formula (XIX) that are preferred include compounds in which R⁷ represents hydrogen, particularly compounds in which R⁸ represents hydrogen, methyl, or fluorine.

Additional compounds of Formula (XIX) that are preferred include compounds in which R⁸ represents hydrogen. Additional compounds of Formula (XIX) that are preferred include compounds in which Ar represents phenyl that may be substituted by 1 or 2 substituents, wherein the substituents are selected independently from one another from the group consisting of methyl, methoxy, fluorine, and chlorine.

Additional derivatives and analogs of letermovir are disclosed in U.S. Pat. No. 7,960,387 by Wunberg et al., incorporated herein by this reference. These derivatives and analogs include, but are not limited to, compounds of Formula

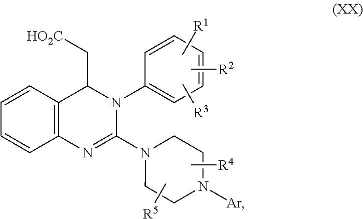

wherein:
(1) Ar represents aryl which may be substituted by 1 to 3 substituents, where the substituents are selected independently of one another from the group consisting of alkyl, alkoxy, formyl, carboxyl, alkylcarbonyl, alkoxycarbonyl, trifluoromethyl, halogen, cyano, hydroxyl, amino, alkylamino, aminocarbonyl and nitro, where alkyl may be substituted by 1 to 3 substituents, where the substituents are selected independently of one another from the group consisting of halogen, amino, alkylamino, hydroxyl and aryl, or two of the substituents on the aryl radical together with the carbon atoms to which they are attached form a 1,3-dioxolane, a cyclopentane ring or a cyclohexane ring, and any third substituent present is selected independently therefrom from the group mentioned;
(2) R¹ represents hydrogen, alkyl, alkoxy, cyano, halogen, nitro or trifluoromethyl;
(3) R² represents hydrogen, alkyl, alkoxy, cyano, halogen, nitro or trifluoromethyl;
(4) R³ represents alkyl, alkoxy, cyano, halogen, nitro or trifluoromethyl; or
(5) one of the radicals R¹, R² and R³ represents hydrogen, alkyl, alkoxy, cyano, halogen, nitro or trifluoromethyl and the other two together with the carbon atoms to which they are attached form a 1,3-dioxolane, a cyclopentane ring or a cyclohexane ring;
(6) R⁴ represents hydrogen or alkyl;
(7) R⁵ represents hydrogen or alkyl; or
(8) the radicals R⁴ and R⁵ are attached to carbon atoms directly opposing each other in the piperazine ring and form a methylene bridge which is optionally substituted by 1 or 2 methyl groups;
   and the salts of these compounds, solvates of these compounds, and solvates of salts of these compounds.

For substituents in compounds of Formula (XX), the substituents “alkyl,” “alkoxy,” “alkylamino,” “alkylcarbonyl,” “alkoxycarbonyl,” “aryl,” and “halogen” are defined as for corresponding substituents in compounds of Formula (XIX), above.

For compounds of Formula (XX), physiologically acceptable salts include the same acid addition salts and salts of bases as for compounds of Formula (XIX), above.

Compounds of Formula (XX) are preferred in which Ar represents phenyl which may be substituted by 1 to 3 substituents, where the substituents are selected independently of one another from the group consisting of C₁-C₆-alkyl, C₁-C₆-alkoxy, carboxyl, C₁-C₆-alkylcarbonyl, C₁-C₆-alkoxycarbonyl, trifluoromethyl, fluorine, chlorine, bromine, cyano, hydroxyl, amino, C₁-C₆-alkylamino and nitro, or two of the substituents on the aryl radical together with the carbon atoms to which they are attached form a 1,3-dioxolane and any third substituent present is selected independently therefrom from the group mentioned; R¹ represents hydrogen, C₁-C₃-alkyl, C₁-C₃-alkoxy, fluorine or chlorine, R² represents hydrogen, C₁-C₃-alkyl, C₁-C₃-alkoxy, fluorine or chlorine; R³ represents C₁-C₄-alkyl, cyano, fluorine, chlorine, nitro or trifluoromethyl; R⁴ represents hydrogen or methyl; and R⁵ represents hydrogen.

Among these, particular preference is given to those compounds of Formula (XX), in which Ar represents phenyl which may be substituted by 1 or 2 substituents, where the substituents are selected independently of one another from the group consisting of methyl, methoxy, fluorine and chlorine; R¹ represents hydrogen, methyl, methoxy, fluorine or chlorine; R² represents hydrogen; R³ represents methyl, isopropyl, t-butyl, cyano, fluorine, chlorine, nitro or trifluoromethyl; R⁴ represents hydrogen; and R⁵ represents hydrogen.

Additionally preferred compounds of Formula (XX) are those compounds in which R¹ represents hydrogen, methyl, methoxy, or fluorine.

Additionally preferred compounds of Formula (XX) are those compounds in which R¹ is attached to the phenyl ring via the position ortho to the point of attachment of the phenyl ring. For these compounds of Formula (XX) as used herein in compositions and methods according to the present invention, the point of attachment of the phenyl ring substituted by radicals R¹, R², and R³ is to be understood as meaning the carbon atom of the phenyl ring which, according to Formula (XX), is attached to one of the two nitrogen atoms of the dihydroquinazoline. Among these compounds are compounds of Formula (XX) in which R¹ represents methyl or methoxy and R¹ is attached to the phenyl ring via the position ortho to the point of attachment of the phenyl ring.

Additionally preferred compounds of Formula (XX) are those compounds in which R² represents hydrogen.

Additionally preferred compounds of Formula (XX) are those compounds in which R³ represents trifluoromethyl, chlorine, methyl, isopropyl or t-butyl.

Additionally preferred compounds of Formula (XX) are those compounds in which R¹ is attached to the phenyl ring via the position ortho to the point of attachment of the phenyl ring and R³ is attached to the phenyl ring via the position meta to the point of attachment of the phenyl ring, which position is opposite to that of R¹. In these compounds, R³ is preferably trifluoromethyl, chlorine, or methyl.

Additionally preferred compounds of Formula (XX) are those compounds in which R⁴ and R⁵ represent hydrogen.

Additionally preferred compounds of Formula (XX) are those compounds in which Ar represents phenyl which may be substituted by 1 or 2 substituents, where the substituents are selected independently of one another from the group consisting of methyl, methoxy, fluorine and chlorine.

Additional derivatives and analogs of letermovir are disclosed in U.S. Pat. No. 8,198,282 by Wunberg et al., incorporated herein by this reference. These derivatives and analogs include, but are not limited to, compounds of Formula (XXI):

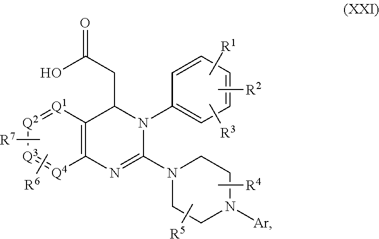

wherein:
(1) Ar is aryl, in which aryl may be substituted by 1 to 3 substituents, where the substituents are selected independently of one another from the group consisting of alkyl, alkoxy, formyl, hydroxycarbonyl, alkylcarbonyl, alkoxycarbonyl, trifluoromethyl, halogen, cyano, hydroxy, amino, alkylamino, aminocarbonyl and nitro, in which alkyl may be substituted by 1 to 3 substituents, where the substituents are selected independently of one another from the group consisting of halogen, amino, alkylamino, hydroxy and aryl, or two of the substituents on the aryl form together with the carbon atoms to which they are bonded a 1,3-dioxolane, a cyclopentane ring or a cyclohexane ring, and an optionally present third substituent is selected independently thereof from the said group;
(2) Q¹, Q², Q³ and Q⁴ are CH or N, where one or two of Q¹, Q², Q³ and Q⁴ are N and the others are simultaneously CH;
(3) R¹ is hydrogen, amino, alkyl, alkoxy, alkylamino, alkylthio, cyano, halogen, nitro or trifluoromethyl;
(4) R² is hydrogen, alkyl, alkoxy, alkylthio, cyano, halogen, nitro or trifluoromethyl;
(5) R³ is amino, alkyl, alkoxy, alkylamino, alkylthio, cyano, halogen, nitro, trifluoromethyl, alkylsulfonyl or alkylaminosulfonyl; or
(6) one of the radicals R¹, R² and R³ is hydrogen, alkyl, alkoxy, cyano, halogen, nitro or trifluoromethyl, and the other two form together with the carbon atoms to which they are bonded a 1,3-dioxolane, a cyclopentane ring or a cyclohexane ring;
(7) R⁴ is hydrogen or alkyl;
(8) R⁵ is hydrogen or alkyl; or
(9) the radicals R⁴ and R⁵ in the piperazine ring are bonded to exactly opposite carbon atoms and form a methylene bridge optionally substituted by 1 to 2 methyl groups;
(10) R⁶ is hydrogen, alkyl, alkoxy, alkylthio, formyl, hydroxycarbonyl, aminocarbonyl, alkylcarbonyl, alkoxycarbonyl, trifluoromethyl, halogen, cyano, hydroxy or nitro; and
(11) R⁷ is hydrogen, alkyl, alkoxy, alkylthio, formyl, hydroxycarbonyl, alkylcarbonyl, alkoxycarbonyl, trifluoromethyl, halogen, cyano, hydroxy or nitro; and the salts of these compounds, solvates of these compounds, and solvates of salts of these compounds.

For substituents in compounds of Formula (XXI), the substituents “alkyl,” “alkoxy,” “alkylamino,” “alkylcarbonyl,” “alkoxycarbonyl,” “aryl,” and “halogen” are defined as for corresponding substituents in compounds of Formula (XIX), above.

For compounds of Formula (XXI), physiologically acceptable salts include the same acid addition salts and salts of bases as for compounds of Formula (XIX), above.

Compounds of Formula (XXI) are preferred in which Ar is phenyl, in which phenyl may be substituted by 1 to 3 substituents, where the substituents are selected independently of one another from the group consisting of C₁-C₆-alkyl, C₁-C₆-alkoxy, hydroxycarbonyl, C₁-C₆-alkylcarbonyl, C₁-C₆-alkoxycarbonyl, trifluoromethyl, fluorine, chlorine, bromine, cyano, hydroxy, amino, C₁-C₆-alkylamino and nitro, or two of the substituents on the phenyl form together with the carbon atoms to which they are bonded a 1,3-dioxolane, and an optionally present third substituent is selected independently thereof from the said group; Q¹, Q² and Q³ are CH or N, where always exactly one of Q¹, Q² and Q³ is N and the others are simultaneously CH, Q⁴ is CH, R¹ is hydrogen, C₁-C₃-alkyl, C₁-C₃-alkoxy, C₁-C₃-alkylthio, fluorine or chlorine; R² is hydrogen, C₁-C₃-alkoxy, C₁-C₃-alkylthio, fluorine or chlorine; R³ is C₁-C₄-alkyl, cyano, fluorine, chlorine, nitro, trifluoromethyl or C₁-C₃-alkylsulfonyl, or one of the radicals R¹, R² and R³ is hydrogen, C₁-C₃-alkyl, C₁-C₃-alkoxy, cyano, halogen, nitro or trifluoromethyl, and the other two form together with the carbon atoms to which they are bonded a cyclopentane ring or a cyclohexane ring; R⁴ is hydrogen or methyl; R⁵ is hydrogen; R⁶ is hydrogen, C₁-C₃-alkyl, C₁-C₃-alkoxy, hydroxycarbonyl, aminocarbonyl, trifluoromethyl, fluorine, chlorine, cyano, hydroxy or nitro; and R⁷ is hydrogen, C₁-C₃-alkyl, C₁-C₃-alkoxy, fluorine, chlorine, cyano or hydroxy.

Among these, particular preference is given to those compounds of Formula (XXI), in which Ar is phenyl, in which phenyl may be substituted by 1 to 2 substituents, where the substituents are selected independently of one another from the group consisting of methyl, methoxy, fluorine and chlorine; Q¹, Q² and Q³ is CH or N, where always exactly one of Q¹, Q² and Q³ is N, and the others are simultaneously CH; Q⁴ is CH; R¹ is hydrogen, methyl, methoxy, methylthio, fluorine or chlorine; R² is hydrogen; R³ is methyl, isopropyl, t-butyl, cyano, fluorine, chlorine, nitro or trifluoromethyl; R⁴ is hydrogen; R⁵ is hydrogen; R⁶ is hydrogen, aminocarbonyl, fluorine, chlorine, cyano or hydroxy; and R⁷ is hydrogen.

Additionally preferred compounds of Formula (XXI) are compounds in which R¹ is hydrogen, methyl, methoxy, or fluorine.

Additionally preferred compounds of Formula (XXI) are compounds in which R¹ is bonded to the phenyl ring via the position ortho to the point of linkage of the phenyl ring. For these compounds of Formula (XXI) as used herein in compositions and methods according to the present invention, the point of linkage of the phenyl ring substituted by the radicals R¹, R² and R³ means the carbon atom of the phenyl ring which is linked to one of the two dihydroquinazoline nitrogen atoms according to Formula (XXI). Among these compounds are compounds of Formula (XXI) in which R¹ is methoxy, and R¹ is bonded to the phenyl ring via the position ortho to the point of linkage of the phenyl ring.

Additionally preferred compounds of Formula (XXI) are compounds in which R² is hydrogen.

Additionally preferred compounds of Formula (XXI) are compounds in which R³ is trifluoromethyl, chlorine, methyl, isopropyl, or t-butyl.

Additionally preferred compounds of Formula (XXI) are compounds in which R¹ is bonded to the phenyl ring via the position ortho to the point of linkage of the phenyl ring, R³ is trifluoromethyl, chlorine or methyl, and R³ is bonded to the phenyl ring via the position opposite to R¹ and meta to the point of linkage of the phenyl ring.

Additionally preferred compounds of Formula (XXI) are compounds in which R⁴ and R⁵ are hydrogen.

Additionally preferred compounds of Formula (XXI) are compounds in which R⁶ is hydrogen.

Additionally preferred compounds of Formula (XXI) are compounds in which R⁷ is hydrogen.

Additionally preferred compounds of Formula (XXI) are compounds in which Ar is phenyl, in which phenyl may be substituted by 1 to 2 substituents, where the substituents are selected independently of one another from the group consisting of methyl, methoxy and fluorine and chlorine.

Additional derivatives and analogs of letermovir are disclosed in U.S. Pat. No. 8,314,113 by Wunberg et al., incorporated herein by this reference. These derivatives and analogs include, but are not limited to, compounds of Formula (XXII):

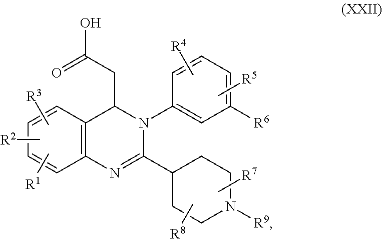

wherein:
(1) R¹, R² and R³ independently of one another represent hydrogen, alkyl, alkoxy, carboxyl, alkylcarbonyl, alkoxycarbonyl, aminocarbonyl, trifluoromethyl, halogen, cyano, hydroxyl or nitro;
(2) R⁴ and R⁵ independently of one another represent hydrogen, alkyl, alkoxy, alkylthio, cyano, halogen, nitro, trifluoromethyl or trifluoromethoxy;
(3) R⁶ represents alkyl, cyano, halogen, nitro or trifluoromethyl;
(4) R⁷ and R⁸ independently of one another represent hydrogen, halogen, alkyl or alkoxy; and
(5) R⁹ represents aryl or 1,3-benzodioxol-5-yl, where aryl and 1,3-benzodioxol-5-yl may be substituted by 1 to 3 substituents, where the substituents independently of one another are selected from the group consisting of alkoxy, alkylthio, carboxyl, alkylcarbonyl, alkoxycarbonyl, am inocarbonyl, trifluoromethyl, halogen, carbamoyl, cyano, hydroxyl, amino, alkylamino, nitro and optionally hydroxyl-substituted alkyl; and the salts of these compounds, solvates of these compounds, and solvates of salts of these compounds.

For substituents in compounds of Formula (XXII), the substituents “alkyl,” “alkoxy,” “alkylamino,” “alkylcarbonyl,” “alkoxycarbonyl,” “aryl,” and “halogen” are defined as for corresponding substituents in compounds of Formula (XIX), above.

For compounds of Formula (XXII), physiologically acceptable salts include the same acid addition salts and salts of bases as for compounds of Formula (XIX), above.

Compounds of Formula (XXII) are preferred in which R¹, R² and R³ independently of one another represent hydrogen, methyl, fluorine, chlorine, cyano, hydroxyl or aminocarbonyl; R⁴ and R⁵ independently of one another represent hydrogen, fluorine, alkyl or alkoxy; R⁶ represents chlorine, nitro, trifluoromethyl, methyl, isopropyl or t-butyl; R⁷ and R⁸ independently of one another represent hydrogen or C₁-C₃-alkyl and R⁹ represents phenyl or 1,3-benzodioxol-5-yl, where phenyl may be substituted by 1 to 3 substituents, where the substituents independently of one another are selected from the group consisting of C₁-C₆-alkyl, C₁-C₆-alkoxy, carboxyl, C₁-C₆-alkylcarbonyl, C₁-C₆-alkoxycarbonyl, trifluoromethyl, fluorine, chlorine, bromine, cyano, hydroxyl, amino, C₁-C₆-alkylamino and nitro.

Additionally preferred compounds of Formula (XXII) are compounds in which R¹ and R² represent hydrogen; R³ represents fluorine; R⁴ and R⁵ independently of one another represent hydrogen, fluorine or alkoxy; R⁶ represents trifluoromethyl; R⁷ and R⁸ represent hydrogen, and R⁹ represents phenyl, where phenyl may be substituted by 1 or 2 substituents, where the substituents independently of one another are selected from the group consisting of methyl, methoxy, fluorine and chlorine.

Additionally preferred compounds of Formula (XXII) are compounds in which R¹ and R² represent hydrogen.

Additionally preferred compounds of Formula (XXII) are compounds in which R³ is attached to the carbon atom in position 6 or position 8 of the quinazoline skeleton or in which R³ is fluorine, in particular fluorine attached to the carbon atom in position 8 of the quinazoline skeleton.

Additionally preferred compounds of Formula (XXII) are compounds in which R⁴ represents hydrogen and R⁵ represents hydrogen, fluorine, or alkoxy, such as methoxy.

Additionally preferred compounds of Formula (XXII) are compounds in which R⁶ represents trifluoromethyl, isopropyl, methyl, or t-butyl.

Additionally preferred compounds of Formula (XXII) are compounds in which R⁷ and R⁸ represent hydrogen.

Additionally preferred compounds of Formula (XXII) are compounds in which: (i) R⁹ represents phenyl, where phenyl may be substituted by 1 or 2 substituents, where the substituents independently of one another are selected from the group consisting of methyl, methoxy, fluorine and chlorine; (ii) R⁹ represents phenyl, where phenyl is substituted by fluorine in the para-position to the point of attachment to the piperidine ring; (iii) R⁹ represents phenyl, where phenyl is substituted by chlorine, methyl or methoxy in the meta-position to the point of attachment to the piperidine ring; or (iv) R⁹ represents phenyl, where phenyl is substituted by methyl in the meta-position to the point of attachment to the piperidine ring and by fluorine in the para-position to the point of attachment to the piperidine ring.

Additional derivatives and analogs of letermovir are disclosed in U.S. Pat. No. 8,343,981 by Wunberg et al., incorporated herein by this reference. These derivatives and analogs include, but are not limited to, compounds of Formula (XXIII):

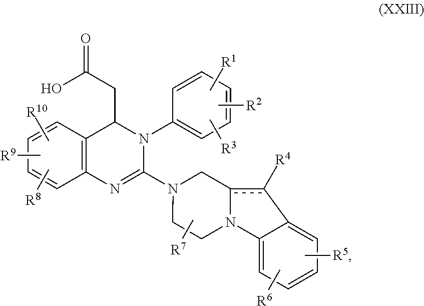

wherein:
(1) [symbol] represents a single or double bond;
(2) R¹ represents hydrogen, amino, alkyl, alkoxy, alkylamino, alkylthio, cyano, halogen, nitro or trifluoromethyl;
(3) R² represents hydrogen, alkyl, alkoxy, alkylthio, cyano, halogen, nitro or trifluoromethyl;
(4) R³ represents amino, alkyl, alkoxy, alkylamino, alkylthio, cyano, halogen, nitro, trifluoromethyl, alkylsulfonyl or alkylaminosulfonyl; or
(5) one of the radicals R¹, R² and R³ represents hydrogen, alkyl, alkoxy, cyano, halogen, nitro or trifluoromethyl and the other two together with the carbon atoms to which they are attached form a 1,3-dioxolane, a cyclopentane ring or a cyclohexane ring;
(6) R⁴ represents hydrogen or alkyl;
(7) R⁵ represents hydrogen, alkyl, alkoxy, formyl, carboxyl, alkylcarbonyl, alkoxycarbonyl, trifluoromethyl, halogen, cyano, hydroxyl, amino, alkylamino, aminocarbonyl or nitro, where alkyl may be substituted by 1 to 3 substituents, where the substituents are selected independently of one another from the group consisting of halogen, amino, alkylamino, hydroxyl and aryl;
(8) R⁶ represents hydrogen, alkyl, alkoxy, formyl, carboxyl, alkylcarbonyl, alkoxycarbonyl, trifluoromethyl, halogen, cyano, hydroxyl, amino, alkylamino, aminocarbonyl or nitro, where alkyl may be substituted by 1 to 3 substituents, where the substituents are selected independently of one another from the group consisting of halogen, amino, alkylamino, hydroxyl and aryl; or
(9) R⁵ and R⁶ together with the carbon atoms to which they are attached form a 1,3-dioxolane, a cyclopentane ring or a cyclohexane ring;
(10) R⁷ represents hydrogen or alkyl;
(11) R⁸ represents hydrogen, alkyl, alkoxy, alkylamino, alkylthio, formyl, carboxyl, am inocarbonyl, alkylaminocarbonyl, alkylcarbonyl, alkoxycarbonyl, trifluoromethyl, halogen, cyano, hydroxyl, nitro or a 5- to 7-membered heterocycle which is attached via nitrogen;
(12) R⁹ represents hydrogen, alkyl, alkoxy, alkylthio, formyl, carboxyl, alkylcarbonyl, alkoxycarbonyl, trifluoromethyl, halogen, cyano, hydroxyl or nitro; and
(13) R¹⁹ represents hydrogen, alkyl, alkoxy, alkylthio, formyl, carboxyl, alkylcarbonyl, alkoxycarbonyl, trifluoromethyl, halogen, cyano, hydroxyl or nitro; and the salts of these compounds, solvates of these compounds, and solvates of salts of these compounds.

For substituents in compounds of Formula (XXIII), the substituents “alkyl,” “alkoxy,” “alkylamino,” “alkylcarbonyl,” “alkoxycarbonyl,” “aryl,” and “halogen” are defined as for corresponding substituents in compounds of Formula (XIX), above. In the compounds of Formula (XXIII), a 5- to 7-membered heterocycle which is attached via nitrogen is a monocyclic non-aromatic heterocycle which is attached via nitrogen and generally has 5 to 7, preferably 5 or 6, ring atoms and up to 2, preferably up to 1, additional heteroatom and/or hetero group from the group consisting of N, O, S, SO, and SO₂. The heterocycle may be saturated or partially unsaturated. Preference is given to 5- or 6-membered monocyclic saturated heterocycles having up to one additional heteroatom from the group consisting of O, N and S, such as, by way of example and preferably, pyrrolidinyl, piperidinyl, piperazinyl, morpholinyl and thiomorpholinyl.

For compounds of Formula (XXIII), physiologically acceptable salts include the same acid addition salts and salts of bases as for compounds of Formula (XIX), above.

Compounds of Formula (XXIII) are preferred in which: [symbol] represents a single or double bond; R¹ represents hydrogen, C₁-C₆-alkyl, C₁-C₆-alkoxy, C₁-C₆-alkylthio, fluorine or chlorine; R² represents hydrogen, C₁-C₆-alkyl, C₁-C₆-alkoxy, C₁-C₆-alkylthio, fluorine or chlorine; R³ represents C₁-C₄-alkyl, cyano, fluorine, chlorine, nitro or trifluoromethyl, or one of the radicals R¹, R² and R³ represents hydrogen, C₁-C₃-alkyl, C₁-C₃-alkoxy, cyano, halogen, nitro or trifluoromethyl and the other two together with the carbon atoms to which they are attached form a cyclopentane ring or a cyclohexane ring; R⁴ represents hydrogen, R⁵ represents hydrogen, C₁-C₆-alkyl, C₁-C₆-alkoxy, carboxyl, C₁-C₆-alkylcarbonyl, C₁-C₆-alkoxycarbonyl, trifluoromethyl, fluorine, chlorine, bromine, cyano, hydroxyl, amino, C₁-C₆-alkylamino or nitro; R⁶ represents hydrogen, C₁-C₆-alkyl, C₁-C₆-alkoxy, carboxyl, C₁-C₆-alkylcarbonyl, C₁-C₆-alkoxycarbonyl, trifluoromethyl, fluorine, chlorine, bromine, cyano, hydroxyl, amino, C₁-C₆-alkylamino or nitro or R⁵ and R⁶ together with the carbon atoms to which they are attached form a 1,3-dioxolane; R⁷ represents hydrogen or methyl; R⁸ represents C₁-C₃-alkyl, C₁-C₃-alkoxy, carboxyl, am inocarbonyl, C₁-C₃-alkylaminocarbonyl, trifluoromethyl, fluorine, chlorine, cyano, hydroxyl or nitro; R⁹ represents hydrogen, C₁-C₃-alkyl, C₁-C₃-alkoxy, fluorine, chlorine, cyano or hydroxyl; and R¹⁰ represents hydrogen, C₁-C₃-alkyl, C₁-C₃-alkoxy, fluorine, chlorine, cyano or hydroxyl. Of these compounds, compounds of Formula (XXIII) in which [symbol] represents a single or double bond; R¹ represents hydrogen, methyl, methoxy, methylthio, fluorine or chlorine; R² represents hydrogen; R³ represents methyl, cyano, fluorine, chlorine, nitro or trifluoromethyl; R⁴ represents hydrogen; R⁵ represents hydrogen, methyl, methoxy, fluorine or chlorine; R⁶ represents hydrogen, methyl, methoxy, fluorine or chlorine; R⁷ represents hydrogen; R⁸ represents aminocarbonyl, fluorine, chlorine, cyano or hydroxyl; R⁹ represents hydrogen; and R¹⁰ represents hydrogen are particularly preferred.

Additionally preferred compounds of Formula (XXIII) are compounds in which [symbol] represents a single bond and compounds in which R¹ represents hydrogen, methyl, methoxy, or fluorine. Additionally preferred compounds of Formula (XXIII) are compounds in which R¹ is attached to the phenyl ring via the position ortho to the point of attachment of the phenyl ring. In terms of the definition of the compounds of Formula (XXIII) the point of attachment of the phenyl ring substituted by radicals R¹, R² and R³ is to be understood as meaning the carbon atom of the phenyl ring which, according to Formula (XXIII), is attached to one of the two nitrogen atoms of the dihydroquinazoline.

Additionally preferred compounds of Formula (XXIII) are those compounds in which R² represents hydrogen.

Additionally preferred compounds of Formula (XXIII) are those compounds in which R³ represents trifluoromethyl, chlorine, methyl, isopropyl or t-butyl.

Additionally preferred compounds of Formula (XXIII) are those compounds in which R⁴ represents hydrogen.

Additionally preferred compounds of Formula (XXIII) are those compounds in which R⁵ represents hydrogen, methyl, methoxy, fluorine or chlorine.

Additionally preferred compounds of Formula (XXIII) are those compounds in which R⁶ represents hydrogen, methyl, methoxy or fluorine.

Additionally preferred compounds of Formula (XXIII) are those compounds in which R⁷ represents hydrogen.

Additionally preferred compounds of Formula (XXIII) are those compounds in which R⁸ represents fluorine. In such compounds, it is particularly preferred that the fluorine is attached to the aromatic six-membered ring of the dihydroquinazoline moiety as shown in Formula (XXIII(a)):

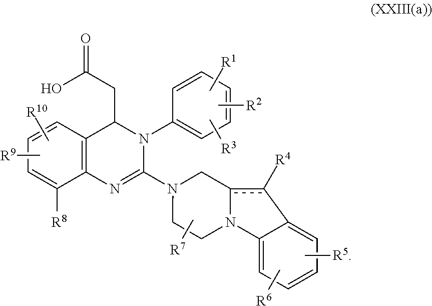

Additionally preferred compounds of Formula (XXIII) are those compounds in which R⁹ represents hydrogen.

Additionally preferred compounds of Formula (XXIII) are those compounds in which R¹⁹ represents hydrogen, methyl or fluorine.

Additional derivatives and analogs of letermovir are disclosed in U.S. Pat. No. 8,513,255 by Wunberg et al., incorporated herein by this reference. These derivatives and analogs include, but are not limited to, compounds of Formula (XXIV):

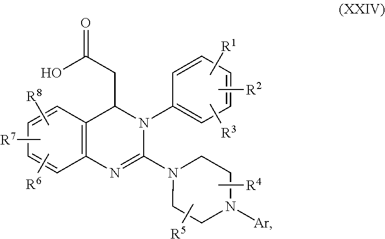

wherein:
(1) Ar represents aryl which may be substituted by 1 to 3 substituents, where the substituents are selected independently of one another from the group consisting of alkyl, alkoxy, formyl, carboxyl, alkylcarbonyl, alkoxycarbonyl, trifluoromethyl, halogen, cyano, hydroxyl, amino, alkylamino, aminocarbonyl and nitro, where alkyl may be substituted by 1 to 3 substituents, where the substituents are selected independently of one another from the group consisting of halogen, amino, alkylamino, hydroxyl and aryl, or two of the substituents on the aryl radical together with the carbon atoms to which they are attached form a 1,3-dioxolane, a cyclopentane ring or a cyclohexane ring, and any third substituent present is selected independently from the group mentioned;
(2) R¹ represents hydrogen, amino, alkyl, alkoxy, alkylamino, alkylthio, cyano, halogen, nitro or trifluoromethyl;
(3) R² represents hydrogen, alkyl, alkoxy, alkylthio, cyano, halogen, nitro or trifluoromethyl;
(4) R³ represents amino, alkyl, alkoxy, alkylamino, alkylthio, cyano, halogen, nitro, trifluoromethyl, alkylsulfonyl or alkylaminosulfonyl; or
(5) one of the radicals R¹, R² and R³ represents hydrogen, alkyl, alkoxy, cyano, halogen, nitro or trifluoromethyl and the other two together with the carbon atoms to which they are attached form a 1,3-dioxolane, a cyclopentane ring or a cyclohexane ring;
(6) R⁴ represents hydrogen or alkyl;
(7) R⁵ represents hydrogen or alkyl; or
(8) the radicals R⁴ and R⁵ are attached to carbon atoms directly opposing each other in the piperazine ring and form a methylene bridge which is optionally substituted by 1 or 2 methyl groups;
(9) R⁶ represents alkyl, alkoxy, alkylthio, formyl, carboxyl, aminocarbonyl, alkylcarbonyl, alkoxycarbonyl, trifluoromethyl, halogen, cyano, hydroxyl or nitro;
(10) R⁷ represents hydrogen, alkyl, alkoxy, alkylthio, formyl, carboxyl, alkylcarbonyl, alkoxycarbonyl, trifluoromethyl, halogen, cyano, hydroxyl or nitro; and
(11) R⁸ represents hydrogen, alkyl, alkoxy, alkylthio, formyl, carboxyl, alkylcarbonyl, alkoxycarbonyl, trifluoromethyl, halogen, cyano, hydroxyl or nitro; and the salts of these compounds, solvates of these compounds, and solvates of salts of these compounds.

For substituents in compounds of Formula (XXIV), the substituents “alkyl,” “alkoxy,” “alkylamino,” “alkylcarbonyl,” “alkoxycarbonyl,” “aryl,” and “halogen” are defined as for corresponding substituents in compounds of Formula (XIX), above.

Compounds of Formula (XXIV) are preferred in which: Ar represents phenyl which may be substituted by 1 to 3 substituents, where the substituents are selected independently of one another from the group consisting of C₁-C₆-alkyl, C₁-C₆-alkoxy, carboxyl, C₁-C₆-alkylcarbonyl, C₁-C₆-alkoxycarbonyl, trifluoromethyl, fluorine, chlorine, bromine, cyano, hydroxyl, amino, C₁-C₆-alkylamino and nitro, or two of the substituents on the phenyl radical together with the carbon atoms to which they are attached form a 1,3-dioxolane and any third substituent present is selected independently from the group mentioned; R¹ represents hydrogen, C₁-C₃-alkyl, C₁-C₃-alkoxy, C₁-C₃-alkylthio, fluorine or chlorine; R² represents hydrogen, C₁-C₃-alkyl, C₁-C₃-alkoxy, C₁-C₃-alkylthio, fluorine or chlorine; R³ represents C₁-C₄-alkyl, cyano, fluorine, chlorine, nitro, trifluoromethyl or C₁-C₃-alkylsulfonyl, or one of the radicals R² and R³ represents hydrogen, C₁-C₃-alkyl, C₁-C₃-alkoxy, cyano, halogen, nitro or trifluoromethyl and the other two together with the carbon atoms to which they are attached form a cyclopentane ring or a cyclohexane ring; R⁴ represents hydrogen or methyl; R⁵ represents hydrogen; R⁶ represents C₁-C₃-alkyl, C₁-C₃-alkoxy, carboxyl, aminocarbonyl, trifluoromethyl, fluorine, chlorine, cyano, hydroxyl or nitro; R⁷ represents hydrogen, C₁-C₃-alkyl, C₁-C₃-alkoxy, fluorine, chlorine, cyano or hydroxyl; and R⁸ represents hydrogen, C₁-C₃-alkyl, C₁-C₃-alkoxy, fluorine, chlorine, cyano or hydroxyl. Of these compounds, compounds in which Ar represents phenyl which may be substituted by 1 or 2 substituents, where the substituents are selected independently of one another from the group consisting of methyl, methoxy, fluorine and chlorine; R¹ represents hydrogen, methyl, methoxy, methylthio, fluorine or chlorine, R² represents hydrogen; R³ represents methyl, isopropyl, t-butyl, cyano, fluorine, chlorine, nitro or trifluoromethyl; R⁴ represents hydrogen; R⁵ represents hydrogen; R⁶ represents aminocarbonyl, fluorine, chlorine, cyano or hydroxyl; R⁷ represents hydrogen; and R⁸ represents hydrogen, fluorine or chlorine are particularly preferred.

Additionally preferred compounds are compounds of Formula (XXIV) in which R¹ represents hydrogen, methyl, methoxy or fluorine.

Additionally preferred compounds are compounds of Formula (XXIV) in which R¹ is attached to the phenyl ring via the position ortho to the point of attachment of the phenyl ring. In terms of the definition of the compounds of Formula (XXIV), the point of attachment of the phenyl ring substituted by radicals R¹, R² and R³ is to be understood as meaning the carbon atom of the phenyl ring which, according to Formula (I), is attached to one of the two nitrogen atoms of the dihydroquinazoline.

Additionally preferred compounds are compounds of Formula (XXIV) in which R² represents hydrogen.

Additionally preferred compounds are compounds of Formula (XXIV) in which R³ represents trifluoromethyl, chlorine, methyl, isopropyl or t-butyl.

Additionally preferred compounds are compounds of Formula (XXIV) in which R¹ is attached to the phenyl ring via the position ortho to the point of attachment of the phenyl ring and R³ is attached to the phenyl ring via the position meta to the point of attachment of the phenyl ring, which position is opposite to that of R¹.

Additionally preferred compounds are compounds of Formula (XXIV) in which R⁴ and R⁵ represent hydrogen.

Additionally preferred compounds are compounds of Formula (XXIV) in which R⁶ represents fluorine. In such compounds, it is particularly preferred that the fluorine is attached to the aromatic six-membered ring of the dihydroquinazoline moiety as shown in Formula (X(a)):

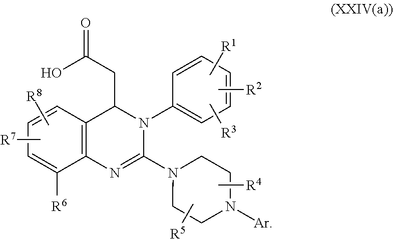

Additionally preferred compounds are compounds of Formula (XXIV) in which R⁷ represents hydrogen.

Additionally preferred compounds are compounds of Formula (XXIV) in which R⁸ represents hydrogen, methyl, or fluorine.

In addition, United States Patent Application Publication No. 2014/0193802 by Lischka, incorporated herein by this reference, discloses a method for the detection of an altered therapeutic response of a subject infected by HCMV to a treatment with a 3,4 dihydroquinazoline or N-{3-[({4-[5-(6-aminopyridin-2-yl)-1,2,4-oxadiazol-3-yl]phenyl}sulfonyl)amino]-5-fluorophenyl}-1-cyanocyclopropanecarboxamide, a method for the detection of a drug resistance of a HCMV to a 3,4-dihydroquinazoline or N-{3-[({4-[5-(6-aminopyridin-2-yl)-1,2,4-oxadiazol-3-yl]phenyl}sulfonyl)amino]-5-fluorophenyl}-1-cyanocyclopropanecarboxamide, and to a method for the detection of a mutation of a HCMV resulting in a drug resistance to a 3,4-dihydroquinazoline or N-{3-[({4-[5-(6-aminopyridin-2-yl)-1,2,4-oxadiazol-3-yl]phenyl}sulfonyl)amino]-5-fluorophenyl}-1-cyanocyclopropanecarboxamide.

Additional derivatives and analogs of letermovir are known in the art.

Prodrugs of letermovir are also known in the art.

Valganciclovir can be used for the treatment of cytomegalovirus or for the treatment of human herpesvirus 4 (Epstein-Barr virus).

Valganciclovir has the IUPAC name 2-[(2-amino-6-oxo-6,9-dihydro-3H-purin-9-yl)methoxy]-3-hydroxypropyl-(2S)-2-amino-3-methylbutanoate. Valganciclovir is described in U.S. Pat. No. 6,083,953 to Nestor et al., incorporated herein by this reference. Methods for synthesis of valganciclovir, specifically, valganciclovir hydrochloride, are disclosed in U.S. Pat. No. 8,586,738 to Hashmi et al., incorporated herein by this reference Valganciclovir is a prodrug of ganciclovir. Prodrugs of vanganciclovir are described in U.S. Pat. No. 8,357,723 by Satyam and in U.S. Pat. No. 8,354,455 by Satyam, both incorporated herein by this reference.

Still other additional therapeutic agents can be used. For example, and not by way of limitation, when the method is for the treatment or prevention of cytomegalovirus infection, additional therapeutic agents can include, but are not limited to, ganciclovir, cidofovir, and foscarnet. When the method is for the treatment or prevention of Epstein-Barr virus infection, additional therapeutic agents can include, but are not limited to, ganciclovir, acyclovir, valaciclovir, cidofovir, adefovir, foscarnet, and romidepsin. When the method is for the treatment or prevention of HSV virus infection, additional therapeutic agents can include, but are not limited to, acyclovir, valaciclovir, famciclovir, and penciclovir. When the method is for the treatment or prevention of HIV virus infection, additional therapeutic agents can include, but are not limited to: nucleoside reverse transcriptase inhibitors, including, but not limited to, zidovudine, didanosine, stavudine, zalcitabine, lamivudine, abacavir, tenofovir disoproxil, and emtrictabine; non-nucleoside reverse transcriptase inhibitors, including, but not limited to, nevirapine, efavirenz, and delavirdine; protease inhibitors, including, but not limited to, saquinivir, indinavir, ritonavir, nelfinavir, amprenavir, lopinavir, atazanavir, and fosamprenavir; and fusion inhibitors, including, but not limited to, enfuvirtide. When the method is for treatment or prevention of HCV virus infection, additional therapeutic agents can include, but are not limited to, sofosbuvir, ribavirin, pegylated interferon-α-2a, pegylated interferon-α-2b, boceprevir, telaprevir, ledipasvir, and simiprevir. Other suitable therapeutic agents are known in the art.

When the method is for the treatment or prevention of Epstein-Barr virus infection, the additional therapeutic agent can be a lytic induction agent that induces the lytic form of the virus. This provides for more efficient elimination of the virus in terms of its existence in a latent form in the genome of infected cells. Lytic induction agents can include, but are not limited to: 5-fluorouracil, cisplatin, taxol, 5-iodo-2′-deoxyuridine, phorbol ester tetradecanoyl phorbol acetate, doxorubicin, gemcitabine, butyrate salts phenylbutyrate, arsenic trioxide, calcium ionophores, 5-azacytidine, 5-aza-2′-deoxycytidine, procaine, trichostatin A, trapoxin B, histone acetylating agents, histone deacetylase inhibitors, dexamethasone, rituximab, depsipeptides, vorinostat, romidepsin, belinostat, suberoylanilide hydroxamic acid, cinnamic acid hydroxamate, panobinostat, entinostat, mocetinostat, abexinostat, pracinostat, resminostat, givinostat, quisinostat, 7-(4-(3-ethynylphenylamino)-7-methoxyquinazolin-6-yloxy)-N-hydroxyheptanamide (CUDC-101), N-hydroxy-4-[[(2S)-3-methyl-2-phenylbutanoyl]amino]benzamide (AR-42), tefinostat, 2-(6-{[(6-fluoroquinolin-2-yl)methyl]amino}bicyclo[3.1.0]hex-3-yl)-N-hydroxypyrimidine-5-carboxamide (CHR-3996), 4SC-202, (E)-N1-(3-(dimethylamino)propyl)-N8-hydroxy-2-((naphthalen-1-yloxy)methyl)oct-2-enediamide (CG200745), rocinilinostat, 4,4′-(7-hydroxy-8-methylchroman-3,4-diyl)diphenol (ME-344), sulforaphane, kevetrin, and valproic acid. In some embodiments, the lytic induction agent induces the expression of BZLF1 and BRLF1 proteins of the Epstein-Barr virus. In still other embodiments, 30 the Epstein-Barr virus lytic induction agent induces the expression of BMRF1, BZLF1 and BRLF1 proteins of the Epstein-Barr virus.

The agents 5-azacytidine, 5-aza-2′-deoxycytidine, and procaine act as demethylating agents; more specifically, they act as DNA methylation inhibitors.

The agents valproic acid, trichostatin A, trapoxin B, phenylbutyrate, depsipeptides, vorinostat, romidepsin, belinostat, suberoylanilide hydroxamic acid, cinnamic acid hydroxamate, panobinostat, entinostat, mocetinostat, abexinostat, pracinostat, resminostat, givinostat, quisinostat, 7-(4-(3-ethynylphenylamino)-7-methoxyquinazolin-6-yloxy)-N-hydroxyheptanamide (CUDC-101), N-hydroxy-4-[[(2S)-3-methyl-2-phenylbutanoyl]amino]benzamide (AR-42), tefinostat, 2-(6-{[(6-fluoroquinolin-2-yl)methyl]amino}bicyclo[3.1.0]hex-3-yl)-N-hydroxypyrimidine-5-carboxamide (CHR-3996), 4SC-202, (E)-N1-(3-(dimethylamino)propyl)-N8-hydroxy-2-((naphthalen-1-yloxy)methyl)oct-2-enediamide (CG200745), rocinilinostat, 4,4′-(7-hydroxy-8-methylchroman-3,4-diyl)diphenol (ME-344), sulforaphane, kevetrin, and valproic acid act as histone deactylase inhibitors. The role of histone deactylase inhibitors in epigenetic modulation and control is disclosed in X.-J. Yang & E. Seto, “HATs and HDACs: From Structure, Function and Regulation to Novel Strategies for Therapy and Prevention,” Oncogene 26: 5310-5318 (2007), incorporated herein by this reference.

Additional antiviral agents that can be used in combination with compounds according to the present invention as described above include, but are not limited to, valomaciclovir stearate, octadecyloxyethyl-cidofovir, hexadecyloxypropyl-cidofovir, adefovir, amantadine, arbidol, brivudine, darunavir, docosanol, edoxudine, entecavir, fomivirsen, fosfonet, ibacitabine, immunovir, idoxuridine, imiquimod, inosine, loviride, raltegravir, maraviroc, moroxydine, nelfinavir, nexavir, oseltamivir, peramivir, pleconaril, podophyllotoxin, rimantidine, tenofovir, tipranavir, trifluridine, tromantidine, vicriviroc, vidarabine, viramidine, zanamivir, (2-amino-7-[(1,3-dihydroxy-2-propoxy)methyl]purine], (1'S,2′R)-9-[[1′,2′-bis(hydroxymethyl)cycloprop-1′-yl]methyl]guanine (A-5021), cyclopropavir, 2,4-diamino-6-R-[3-hydroxy-2(phosphonomethoxy)propoxy]-pyrimidine, (S)-9-(3-hydroxy-2-phosphonylmethoxypropyl)adenine (S-HPMPA), 3-deaza-9-(3-hydroxy-2-phosphonylmethoxypropyl)adenine (3-deaza-HPMPA), N-(4-chlorobenzyl)-1-methyl-6-(4-morpholinylmethyl)-4-oxo-1,4-dihydro-3-quinolinecarboxamine (PNU-183792), 2-bromo-5,6-dichloro-1-(β-D-ribofuranosyl)benzimidazole (BDCRB), maribavir, 3-hydroxy-2,2-dimethyl-N-[4-{[(5-dimethylamino)-1-naphthyl]-sulfonyl]-amino)phenyl}propamide (BAY 38-4766), N—[N-[4-(2-Aminothiazol-4-yl)phenyl]carbamoylmethyl]-N-[1(5)-phenylethyl]pyridine-4-carboxamide (BILS179BS), N-[5-(aminosulfonyl)-4-methyl-1,3-thiazol-2-yl]-N-methyl-2-{4-(2-pyridinyl)phenyl}acetamide (BAY 57-1293), 2H-3-(4-chlorophenyl)-3,4-dihydro-1,4-benzo-thiazine-2-carbonitrile 1,1-dioxide, and 2-chloro-3-pyridin-3-yl-5,6,7,8-tetrahydronindolizine-1-carboxamide (CMV423).

The additional antiviral agent can also be an integrase inhibitor. Integrase inhibitors include, but are not limited to, elvitegravir, dolutegravir, raltegravir, and (6S)-2-(3-chloro-4-fluorobenzyl)-8-ethyl-10-hydroxy-N,6-dimethyl-1,9-dioxo-1,2,6,7,8,9-hexahydropyrazino[1′,2′:1,5]pyrrolo[2,3-d]pyridazine-4-carboxamide (MK-2048).

The additional antiviral agent can also be an entry inhibitor. Entry inhibitors include, but are not limited to, include fostemsavir (BMS-663068), aplaviroc, (5,5′-((1E,1′E)-(methylenedisulfonyl)bis(ethane-2,1-diyl))bis(benzene-1,2,3-triol) (DCM205), and VIR-576.

The additional antiviral agent can also be an interferon, such as, but not limited to, an interferon type I, an interferon type II, and an interferon type III. Other interferons are known in the art. Chemically modified interferons, such as pegylated interferons, are also known in the art.

The additional antiviral agent can also be a synergistic enhancer. Synergistic enhancers are compounds that, by themselves, do not possess antiviral activity or only possess weak antiviral activity, but synergistically increase the activity of other antiviral agents. Synergistic enhancers include, but are not limited to, hydroxyurea, leflunomide, mycophenolic acid, and resveratrol.

As detailed below, compounds according to the present invention can be administered in the form of the compounds themselves, but are typically administered in the form of pharmaceutical compositions including at least one pharmaceutically acceptable carrier.

Pharmaceutically Acceptable Carriers

As stated above, compounds according to the present invention are typically administered in the form of a pharmaceutical composition. A pharmaceutical composition according to the present invention comprises: (1) a compound according to the present invention; and (2) at least one pharmaceutically acceptable carrier. Pharmaceutical compositions according to the present invention can be formulated in dosage forms as described below. These dosage forms can be formulated incorporating specific pharmaceutically acceptable carriers or can be formulated for specific routes of administration as described.

When an additional antiviral agent is employed in a method according to the present invention, the additional antiviral agent can be included in the pharmaceutical composition. The additional antiviral agent is included in the pharmaceutical composition in a therapeutically effective quantity. Alternatively, the additional antiviral agent can be administered separately, either in the form of the additional antiviral agent itself, or in the form of a second pharmaceutical composition including the additional antiviral agent. More than one additional antiviral agent can be administered; if more than one additional antiviral agent is administered, each additional antiviral agent can be administered in the form of the additional antiviral agent itself, or in the form of a pharmaceutical composition. For example, two pharmaceutical compositions can be used, one including a compound according to the present invention and another including both of the additional antiviral agents. Other alternatives are possible with respect to the inclusion of additional antiviral agents in pharmaceutical compositions.

For such pharmaceutical compositions, compounds according to the present invention or additional antiviral agents can be formulated into suitable preparations such as dosage forms that are, for example, solutions, suspensions, tablets, dispersible tablets, pills, capsules, powders, sustained release formulations or elixirs, for oral administration; or in sterile solutions or suspensions for parenteral administration, as well as transdermal patch preparation and dry powder inhalers. In some embodiments, the compounds described above are formulated into compositions using techniques and procedures well known in the art ((see, e.g., Ansel Introduction to Pharmaceutical Dosage Forms, Seventh Edition (1999).

In the compositions, effective concentrations of one or more compounds or derivatives thereof is (are) mixed with a suitable vehicle. The compounds may be derivatized as the corresponding salts, esters, enol ethers or esters, acetals, ketals, orthoesters, hem iacetals, hem iketals, acids, bases, solvates, hydrates or prodrugs prior to formulation, as described above.

Compounds according to the present invention, or additional antiviral compounds as described above, are included in the vehicle in an amount sufficient to exert a therapeutically useful effect in the absence of undesirable side effects on the subject treated. The therapeutically effective concentration may be determined empirically by testing the compounds in in vitro and in vivo systems well known to those of skill in the art and then extrapolated therefrom for dosages for humans. If non-human subjects are to be treated, appropriate calculations to determine suitable dosages can be made, based on weight and factors specific to the metabolism of the particular species to which the compounds according to the present invention or additional antiviral agents are to be administered. Typically, the dosage, whether administered in the form of compounds of the present invention, additional antiviral agents, or pharmaceutical compositions, are from about 0.001 mg to about 2000 mg of compound per kilogram of body weight per day. Dosage unit forms can be prepared, for example, to provide from about 0.01 mg, 0.1 mg or 1 mg to about 500 mg, 1000 mg or 2000 mg, and in some embodiments, from about 10 mg to about 500 mg of the active ingredient or a combination of essential ingredients per dosage unit form. In other embodiments, the compounds may be administered at a daily dose generally in the range 0.1 mg/kg/day to 200 mg/kg/day, 0.5 mg/kg/day to 100 mg/kg/day, 10 mg/kg/day to 50 mg/kg/day or 10 mg/kg/day to 25 mg/kg/day.

In some embodiments, compounds according to the present invention are administered at a daily cumulative dose of between about 500 mg and 3000 mg, in split dosing either BID or TID. In other embodiments, compounds according to the present invention are administered at a dose of between about 1.0 g QD and about 3.0 g BID. In still other embodiments, compounds according to the present invention are administered at a dose of between about 1.5 g and about 2.5 g BID. In still other embodiments, compounds according to the present invention are administered at a dose of about 2.0 g BID.

The concentration of the compound according to the present invention, or of any additional antiviral agents to be administered, in the composition will depend on absorption, inactivation and excretion rates of the active compound, the physicochemical characteristics of the compound, the dosage schedule, and the amount administered as well as other factors known to those of skill in the art, such as, but not limited to, additional pharmacokinetic considerations such as kidney and liver function, the severity of the infection, the age and weight of the subject, the response to the compound according to the present invention or to any additional antiviral agents administered, additional medications being administered, including, but not limited to, additional medications that may either accelerate or retard the metabolism of the compound according to the present invention or any additional antiviral agents administered, the route of administration, and the past medical history of the subject. Exemplary doses can include milligram or microgram amounts of the active compounds per kilogram of subject or sample weight (e.g., from about 1 micrograms per kilogram to about 50 milligrams per kilogram, from about 10 micrograms per kilogram to about 30 milligrams per kilogram, from about 100 micrograms per kilogram to about 10 milligrams per kilogram, or from about 100 microgram per kilogram to about 5 milligrams per kilogram). It may be necessary to use dosages of the active ingredients outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the composition provided herein are also encompassed by the above described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, administration of the same formulation provided herein may be repeated and the administrations may be separated by at least 1 day, 2 days, 3 days, 5 days, 10 days, 15 days, 30 days, 45 days, 2 months, 75 days, 3 months or 6 months.

For compounds according to the present invention, a therapeutically effective dosage should produce a serum concentration of active ingredient of from about 0.1 ng/mL to about 50-100 μg/m L. The compositions, in other embodiments, should provide a dosage of from about 0.001 mg to about 2000 mg of compound per kilogram of body weight per day. Dosage unit forms are prepared to provide from about 0.01 mg, 0.1 mg or 1 mg to about 500 mg, 1000 mg or 2000 mg, and in some embodiments from about 10 mg to about 500 mg of the active ingredient or a combination of essential ingredients per dosage unit form.

Active ingredients to be administered, including compounds according to the present invention and additional antiviral agents, may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using co-solvents, such as dimethylsulfoxide (DMSO), using surfactants, such as TWEEN®, or dissolution in aqueous sodium bicarbonate. Derivatives of the compounds, such as prodrugs of the compounds, may also be used in formulating effective compositions.

Upon mixing or addition of the compound(s), the resulting mixture may be a solution, suspension, emulsion or the like. The form of the resulting mixture depends upon a number of factors, including the intended mode of administration and the solubility of the compound in the selected vehicle. The effective concentration is sufficient for ameliorating the symptoms of the disease, disorder or condition treated and may be empirically determined.

The compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or derivatives thereof. The therapeutically active compounds and derivatives thereof are, in some embodiments, formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required vehicle. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of milliliters. Hence, multiple dose form is a multiple of unit-doses which are not segregated in packaging.

Liquid administrable compositions can, for example, be prepared by dissolving, dispersing, or otherwise mixing an active compound as defined above and optional adjuvants in a vehicle, such as, for example, water, saline, aqueous dextrose, glycerol, glycols, ethanol, and the like, to thereby form a solution or suspension. If desired, the composition to be administered may also contain minor amounts of nontoxic auxiliary substances such as wetting agents, emulsifying agents, solubilizing agents, pH buffering agents and the like, for example, sodium acetate, sodium citrate, cyclodextrin derivatives, sorbitan monolaurate, triethanolamine sodium acetate, triethanolamine oleate and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975 or later editions thereof.

Dosage forms or compositions containing active ingredient in the range of 0.005% to 99.9% with the balance made up from non-toxic carrier may be prepared. Methods for preparation of these compositions are known to those skilled in the art.

In certain embodiments, the compositions are lactose-free compositions containing excipients that are well known in the art and are listed, for example, in the U.S. Pharmacopeia (USP) 25-NF20 (2002). In general, lactose-free compositions contains active ingredients, a binder/filler, and a lubricant in compatible and acceptable amounts. Particular lactose-free dosage forms contain active ingredients, microcrystalline cellulose, pre-gelatinized starch, and magnesium stearate.

Further provided are substantially anhydrous compositions and dosage forms comprising active ingredients, since water can facilitate the degradation of some compounds. For example, the addition of water (e.g., 5%) is widely accepted in the art as a means of simulating long-term storage in order to determine characteristics such as shelf-life or the stability of formulations over time (Carstensen, Drug Stability: Principles & Practice, 2d. Ed., Marcel Dekker, NY, N.Y., 1995, pp. 379-80). In effect, water and heat accelerate the decomposition of some compounds. Thus, the effect of water on a formulation can be of great significance since moisture and/or humidity are commonly encountered during manufacture, handling, packaging, storage, shipment, and use of formulations. Anhydrous compositions and dosage forms provided herein can be prepared using anhydrous or low moisture containing ingredients and low moisture or low humidity conditions. An anhydrous composition should be prepared and stored such that its anhydrous nature is maintained. Accordingly, anhydrous compositions are generally packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Typically, oral dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as for example, capsules, tablets, or troches. The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not 20 limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or acceptable derivative thereof, can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes, colorings and flavors.

Pharmaceutically acceptable carriers are generally known in the art. In general, the criteria for such pharmaceutically acceptable carriers include compatibility with the therapeutically active agents included in any composition, such that the pharmaceutically acceptable carrier does not cause chemical degradation of any therapeutically active agent or inhibit the absorption or biodistribution of any therapeutically active agent.

The particular pharmaceutically acceptable carrier or carriers to be used will depend on factors such as the particular dosage form chosen, the route of administration to be used, the quantities of the therapeutically active agents to be included, and the chemical and physical properties of the therapeutic agents to be included.

In all embodiments, tablets and capsule formulations may be coated as known by those of skill in the art in order to modify or sustain dissolution of the active ingredient. Thus, for example, they may be coated with a conventional enterically digestible coating, such as phenyl salicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Vehicles used in elixirs include solvents. Syrups are concentrated aqueous solutions of a sugar, for example, sucrose, and may contain a preservative. An emulsion is a two-phase system in which one liquid is dispersed in the form of small globules throughout another liquid. Vehicles used in emulsions are non-aqueous liquids, emulsifying agents and preservatives. Suspensions use suspending agents and preservatives. Substances used in non-effervescent granules, to be reconstituted into a liquid oral dosage form, include diluents, sweeteners and wetting agents. Substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such as fruits, and synthetic blends of compounds which produce a pleasant taste sensation.

For a solid dosage form, the solution or suspension, in for example, propylene carbonate, vegetable oils or triglycerides, is in some embodiments encapsulated in a gelatin capsule. Such solutions, and the preparation and encapsulation thereof, are disclosed in U.S. Pat. Nos. 4,328,245; 4,409,239; and 4,410,545, incorporated here by this reference. For a liquid dosage form, the solution, e.g., for example, in a polyethylene glycol, may be diluted with a sufficient quantity of a liquid vehicle, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers and encapsulating these solutions or suspensions in hard or soft gelatin capsule shells. Other useful formulations include those set forth in U.S. Pat. No. RE28,819 and 4,358,603. Briefly, such formulations include, but are not limited to, those containing a compound provided herein, a dialkylated mono- or poly-alkylene glycol, including, but not limited to, 1,2-dimethoxyethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including an acetal. Alcohols used in these formulations are any water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

Parenteral administration, in some embodiments characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or non-aqueous. If administered intravenously, suitable carriers include physiological saline or phosphate buffered saline (PBS), and solutions containing thickening and solubilizing agents, such as glucose, polyethylene glycol, and polypropylene glycol and mixtures thereof.

Vehicles used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other substances.

Examples of aqueous vehicles include Sodium Chloride Injection, Ringer's Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Vehicles also include ethyl alcohol, polyethylene glycol and propylene glycol for water miscible vehicles; and sodium hydroxide, hydrochloric acid, citric acid or lactic acid for pH adjustment.

Illustratively, intravenous or intra-arterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound or compounds to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease or condition to be treated and may be empirically determined.

Active ingredients provided herein can be administered by controlled release means or by delivery devices that are well known to those of ordinary skill in the art. Examples include, but are not limited to, those described in U.S. Pat. No. 3,598,123 to Zaffaroni; U.S. Pat. No. 3,845,770 to Theeuwes et al.; U.S. Pat. No. 3,916,899 to Theeuwes et al.; U.S. Pat. No. 4,008,719 to Theeuwes et al.; U.S. Pat. No. 5,674,533 to Santus et al.; U.S. Pat. No. 5,059,595 to Le Grazie; U.S. Pat. No. 5,591,767 to Mohr et al.; U.S. Pat. No. 5,120,548 to McClelland et al.; U.S. Pat. No. 5,073,543 to Marshall et al.; U.S. Pat. No. 5,639,476 to Oshlack et al.; U.S. Pat. No. 5,354,556 to Sparks et al.; U.S. Pat. No. 5,639,480 to Bodmer et al.; U.S. Pat. No. 5,733,566 to Lewis; U.S. Pat. No. 5,739,108 to Mitchell; U.S. Pat. No. 5,891,474 to Busetti et al.; U.S. Pat. No. 5,922,356 to Koseki et al.; U.S. Pat. No. 5,972,891 to Kamei et al.; U.S. Pat. No. 5,980,945 to Ruiz; U.S. Pat. No. 5,993,855 to Yoshimoto et al.; U.S. Pat. No. 6,045,830 to Igari et al.; U.S. Pat. No. 6,087,324 to Igari et al.; U.S. Pat. No. 6,113,943 to Okada et al.; U.S. Pat. No. 6,197,350 to Yamagata et al.; U.S. Pat. No. 6,248,363 to Patel et al.; U.S. Pat. No. 6,264,970 to Hata et al.; U.S. Pat. No. 6,267,981 to Okamoto et al.; U.S. Pat. No. 6,376,461 to Igari et al.; U.S. Pat. No. 6,419,961 to Igari et al.; U.S. Pat. No. 6,589,548 to Oh et al.; U.S. Pat. No. 6,613,358 to Randolph et al.; U.S. Pat. No. 6,699,500 to Okada et al.; and U.S. Pat. No. 6,740,634 by Saikawa et al., all incorporated herein by this reference. Such dosage forms can be used to provide slow or controlled-release of one or more active ingredients using, for example, hydroxypropylmethyl cellulose, other polymer matrices, gels, permeable membranes, osmotic systems, multilayer coatings, microparticles, nanoparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased subject compliance In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the agent may be administered using intravenous infusion, an implantable osmotic pump, a transdermal patch, liposomes, or other modes of administration. In some embodiments, a pump may be used (see, Sefton, CRC Crit. Ref Biomed. Eng. 14:201 (1987); Buchwald et al., Surgery 88:507 (1980); Saudek et al., N Engl. J. Med. 321:574 (1989). In other embodiments, polymeric materials can be used. In other embodiments, a controlled release system can be placed in proximity of the therapeutic target, i.e., thus requiring only a fraction of the systemic dose (see, e.g., Goodson, Medical Applications of Controlled Release, vol. 2, pp. 115-138 (1984). In some embodiments, a controlled release device is introduced into a subject in proximity of the site of inappropriate immune activation or a tumor. Other controlled release systems are discussed in the review by Langer (Science 249:1527-1533 (1990). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinyl alcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, an antioxidant, a buffer and a bulking agent. In some embodiments, the excipient is selected from dextrose, sorbitol, fructose, corn syrup, xylitol, glycerin, glucose, sucrose and other suitable agent. The solvent may contain a buffer, such as citrate, sodium or potassium phosphate or other such buffer known to those of skill in the art at about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Topical mixtures are prepared as described for local o systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or derivatives thereof may be formulated as aerosols for topical application, such as by inhalation through the mouth or nasal passages (see, e.g., U.S. Pat. No. 4,044,126 to Cook et al., U.S. Pat. No. 4,414,209 to Cook et al., and U.S. Pat. No. 4,364,923 to Cook et al., all of which are incorporated herein by this reference, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will, in some embodiments, have diameters of less than 50 microns, in other embodiments less than 10 microns.

The compounds may be formulated for local or topical application, such as for topical application to the skin or mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

For nasal administration, the preparation may contain an active agent as described above, such as H2G or a derivative or analog thereof, dissolved or suspended in a liquid carrier, in particular, an aqueous carrier, for aerosol application. The carrier may contain solubilizing agents such as propylene glycol, surfactants, absorption enhancers such as lecithin or cyclodextrin, or preservatives.

These solutions, particularly those intended for ophthalmic use, may be formulated as 0.01%-10% isotonic solutions, pH about 5-7, with appropriate salts. Other routes of administration, such as transdermal patches, including iontophoretic and electrophoretic devices, and rectal administration, are also contemplated herein. Transdermal patches, including iontophoretic and electrophoretic devices, are well known to those of skill in the art. For example, such patches are disclosed in U.S. Pat. No. 6,267,983 to Fujii et al., U.S. Pat. No. 6,261,595 to Stanley et al., U.S. Pat. No. 6,256,533 to Yuzhakov et al., U.S. Pat. No. 6,167,301 to Flower et al., U.S. Pat. No. 6,024,975 to D'Angelo et al., U.S. Pat. No. 6,010,715 to Wick et al., U.S. Pat. No. 5,985,317 to Venkateshwaran et al., U.S. Pat. No. 5,983,134 to Ostrow, U.S. Pat. No. 5,948,433 to Burton et al., and U.S. Pat. No. 5,860,957 to Jacobsen et al., all of which are incorporated herein by this reference.

Dosage forms for rectal administration include, but are not limited to, rectal suppositories, capsules and tablets for systemic effect. Rectal suppositories as used herein mean solid bodies for insertion into the rectum which melt or soften at body temperature releasing one or more pharmacologically or therapeutically active ingredients. Substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 g. Tablets and capsules for rectal administration are manufactured using the same substances and by the same methods as for formulations for oral administration.

The compounds provided herein, or derivatives thereof, may also be formulated to be targeted to a particular organ, tissue, receptor, or other area of the body of the subject to be treated, so that the composition is thereby targeted to the particular organ, tissue, receptor, or other area of the body of the subject to be treated. This applies either to H2G or the derivative or analog thereof, or to the additional antiviral agent, if such an additional antiviral agent is present in the composition. Many such targeting methods are well known to those of skill in the art. All such targeting methods are contemplated herein for use in the instant compositions. For non-limiting examples of targeting methods, see, e.g., U.S. Pat. No. 6,316,652 to Steliou, U.S. Pat. No. 6,274,552 to Tamarkin et al., U.S. Pat. No. 6,271,359 to Norris et al., U.S. Pat. No. 6,139,865 to Friend et al., U.S. Pat. No. 6,131,570 to Schuster et al., U.S. Pat. No. 6,120,751 to Unger, U.S. Pat. No. 6,071,495 to Unger et al., U.S. Pat. No. 6,060,082 to Chen et al., U.S. Pat. No. 6,048,736 to Kosak, U.S. Pat. No. 6,039,975 to Shah et al., U.S. Pat. No. 6,004,534 to Langer et al., U.S. Pat. No. 5,985,307 to Hanson et al., U.S. Pat. No. 5,972,366 to Hanson et al., U.S. Pat. No. 5,900,252 to Calanchi et al., U.S. Pat. No. 5,840,674 to Yatvin et al., U.S. Pat. No. 5,759,542 to Gurewich, and U.S. Pat. No. 5,709,874 to Hanson et al., all of which are incorporated herein by this reference. Such targeting methods include, but are not limited to, the conjugation of carrier substances, such as antibodies, hormones, receptor agonists or antagonists, or receptors for antiviral compounds as described herein. As used herein, unless further defined or limited, the term “antibody” encompasses both polyclonal and monoclonal antibodies, as well as genetically engineered antibodies such as chimeric or humanized antibodies of the appropriate binding specificity. As used herein, unless further defined, the term “antibody” also encompasses antibody fragments such as sFv, Fv, Fab, Fab′ and F(ab)′₂ fragments. In many cases, it is preferred to use monoclonal antibodies. Receptors are well known in the art and include G-protein coupled receptors (GPCRs). G-protein coupled receptors (GPCRs) are important signal transducing receptors. The superfamily of G protein coupled receptors includes a large number of receptors. These receptors are integral membrane proteins characterized by amino acid sequences that contain seven hydrophobic domains, predicted to represent the transmembrane spanning regions of the proteins. They are found in a wide range of organisms and are involved in the transmission of signals to the interior of cells as a result of their interaction with heterotrimeric G proteins. They respond to a diverse range of agents including lipid analogues, amino acid derivatives, small molecules such as epinephrine and dopamine, and various sensory stimuli. The properties of many known GPCR are summarized in S. Watson & S. Arkinstall, “The G-Protein Linked Receptor Facts Book” (Academic Press, London, 1994), incorporated herein by this reference. GPCR receptors include, but are not limited to, acetylcholine receptors, β-adrenergic receptors, 13₃-adrenergic receptors, serotonin (5-hydroxytryptamine) receptors, dopamine receptors, adenosine receptors, angiotensin Type II receptors, bradykinin receptors, calcitonin receptors, calcitonin gene-related receptors, cannabinoid receptors, cholecystokinin receptors, chemokine receptors, cytokine receptors, gastrin receptors, endothelin receptors, γ-aminobutyric acid (GABA) receptors, galanin receptors, glucagon receptors, glutamate receptors, luteinizing hormone receptors, choriogonadotrophin receptors, follicle-stimulating hormone receptors, thyroid-stimulating hormone receptors, gonadotrophin-releasing hormone receptors, leukotriene receptors, Neuropeptide Y receptors, opioid receptors, parathyroid hormone receptors, platelet activating factor receptors, prostanoid (prostaglandin) receptors, somatostatin receptors, thyrotropin-releasing hormone receptors, vasopressin and oxytocin receptors. Agonists and antagonists specifically binding these receptors can be used as individual carrier substances; suitable receptors, agonists, or antagonists can be selected based on their specificity and the location of the receptors in particular cells or tissues.

If the pharmaceutical composition comprises both H2G or a derivative or analog thereof and an additional antiviral agent, in one alternative, the targeting method targets the H2G or the derivative or analog thereof. In another alternative, the targeting method targets the additional antiviral agent. In another alternative, both the H2G or the derivative or analog thereof and the additional antiviral agent are targeted; the H2G or the derivative or analog thereof and the additional antiviral agent can be targeted by the same targeting method, or, alternatively, the H2G or the derivative or analog thereof and the additional antiviral agent can be targeted by different targeting method.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The compounds or derivatives thereof may be packaged as articles of manufacture containing packaging material, a compound or pharmaceutically acceptable derivative thereof provided herein, which is effective for treatment, prevention or amelioration of one or more symptoms of diseases or disorders associated with viral infection, within the packaging material, and a label that indicates that the compound, derivative or composition thereof, is used for the treatment, prevention or amelioration of one or more symptoms of diseases or disorders associated with viral infection as described above. Specific viral infections can be recited on the label.

The articles of manufacture provided herein contain packaging materials. Packaging materials for use in packaging products are well known to those of skill in the art. See, e.g., U.S. Pat. Nos. 5,323,907, 5,052,558 and 5,033,252, incorporated herein by this reference. Examples of packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder associated with viral infection as described above.

Compounds according to the present invention and pharmaceutical compositions according to the present invention, with the administration of additional antiviral agents as suitable or other suitable agents, can be used to treat or prevent conditions such as systemic sclerosis, myalgic encephalomyelitis/chronic fatigue syndrome, Alzheimer's disease, systemic lupus erythematosus, multiple sclerosis or Graves' disease. For these purposes, therapeutically effective amounts of the compounds or compositions containing therapeutically effective concentrations of the compounds are administered to a patient suffering from systemic sclerosis, myalgic encephalomyelitis/chronic fatigue syndrome, Alzheimer's disease, systemic lupus erythematosus, multiple sclerosis and Graves' disease. As indicated above, there is evidence connecting Alzheimer's disease to the presence of infection by human herpes simplex virus 1 (HSV-1, HHV-1), so that methods and compositions for treating human herpes simplex virus 1 infection have utility for prevention or treatment of Alzheimer's disease.

In another alternative, compounds according to the present invention and pharmaceutical compositions according to the present invention, with the administration of additional antiviral agents as suitable, can be used to treat cancer, particularly cancer associated with the Epstein-Barr virus, in which case the tumor cells typically contain the genome of the Epstein-Barr virus. For example, the cancer can be gastric carcinoma, lymphoma, Hodgkin's disease, nasopharygeal carcinoma, breast cancer, lung cancer, colon cancer, or prostate cancer. If the cancer is lymphoma, the lymphoma can be B-cell lymphoma, an AIDS-related lymphoma, or Burkitt's lymphoma.

Compounds according to the present invention and the pharmaceutical compositions described herein may also be used in combination with one or more other active ingredients. In certain embodiments, the compounds may be administered in combination, or sequentially, with another therapeutic agent. Such other therapeutic agents include those known for treatment of one or more symptoms associated with cancer, systemic sclerosis, myalgic encephalomyelitis/chronic fatigue syndrome, Alzheimer's disease, systemic lupus erythematosus, multiple sclerosis and Graves' disease. These agents are well known in the art.

Suitable agents for treatment of cancer include, but are not limited to, mechlorethamine, cyclophosphamide, ifosfamide, melphalan, chlorambucil, hexamethylmelamine, thiotepa, busulfan, carmustine, streptozocin, dacarbazine, temozolomide, methotrexate, 5-fluorouracil, cytarabine, gemcitabine, 6-mercaptopurine, 6-thioguanine, pentostatin, vinblastine, vincristine, paclitaxel, docetaxel, topotecan, irinotecan, dactinomycin, daunorubicin, doxorubicin, bleomycin, mitomycin C, L-asparaginase, interferon-alfa, interleukin-2, cisplatin, carboplatin, mitoxantrone, hydroxyurea, N-methylhydrazine, mitotane, aminoglutethimide, imatinib, prednisone, prednisolone, methylprednisolone, dexamethasone, betamethasone, triamcinolone, hydroxyprogesterone, medroxyprogesterone, megestrol acetate, diethylstilbestrol, ethinyl estradiol, tamoxifen, anastrozole, testosterone propionate, fluoxymesterone, flutamine, leuprolide, trastuzumab, rituximab, alemtuzumab, bevacizumab, cetuximab, gemtuzumab, ibritumomab, panitumumab, tositumomab, and an interferon.

Suitable agents for treatment of systemic sclerosis include, but are not limited to, non-steroidal anti-inflammatory drugs, steroids including prednisone, calcium channel blockers including nifedipine, iloprost, bosentan, methotrexate, ciclosporin, penicillamine, angiotensin converting enzyme inhibitors, cyclophosphamide, epoprostenol, antithymocyte globulin, and mycophenolate mofetil.

Suitable agents for treatment of myalgic encephalomyelitis/chronic fatigue syndrome include, but are not limited to, antidepressants and immune system stimulating agents.

Suitable agents for treatment of Alzheimer's disease include, but are not limited to, tacrine, rivastigmine, galantamine, donezepil, memantine, and huperzine A.

Suitable agents for treatment of systemic lupus erythematosus include, but are not limited to, non-steroidal anti-inflammatory drugs, prednisone, hydroxychloroquine, methotrexate, azathioprine, cyclophosphamide, mycophenolic acid, belimumab, atacicept, lupuzor, intravenous immunoglobulin, infliximab, anakinra, rituximab, tocilizumab, abatacept, and leflunomide.

Suitable agents for treatment of multiple sclerosis include, but are not limited to, interferon-β1a, interferon-β1b, glatiramer acetate, mitoxantrone, natalizumab, fingolimod, and dimethyl fumarate.

Suitable agents for treatment of idiopathic pulmonary fibrosis include, but are not limited to, pirfenidone, nitedanib, simtuzumab, tralokimab, lebrikizumab, and FG-3019.

Suitable agents for treatment of Graves' disease include, but are not limited to, carbimazole, methimazole, and propylthiouracil.

### Advantages of the Present Invention

The present invention provides compositions and methods to improve treatment of viral infections, especially CMV, HSV, and EPV. The compositions and methods of the present invention provide efficient methods to treat such infections and conditions associated with such infections, including, but not limited to, systemic sclerosis, myalgic encephalomyelitis/chronic fatigue syndrome, Alzheimer's disease, systemic lupus erythematosus, multiple sclerosis or Graves' disease, as well as cancer. These compositions and methods are well-tolerated and can be used together with other therapeutic agents or methods for treatment of such viral infections.

Methods according to the present invention possess industrial applicability for the preparation of a medicament for the treatment of a viral disease in subjects, whether human, or animal, in need of such treatment. Compositions according to the present invention possess industrial applicability as pharmaceutical compositions with a therapeutic use.

The method claims of the present invention provide specific method steps that are more than general applications of laws of nature and require that those practicing the method steps employ steps other than those conventionally known in the art, in addition to the specific applications of laws of nature recited or implied in the claims, and thus confine the scope of the claims to the specific applications recited therein. In some contexts, these claims are directed to new ways of using an existing drug.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

What is claimed is:

1. A method for treatment or prevention of a viral infection comprising the step of administering a therapeutically effective quantity of 9-(4-hydroxy-2-(hydroxymethyl)butyl)guanine (“H2G”) having the structure of Formula (I) 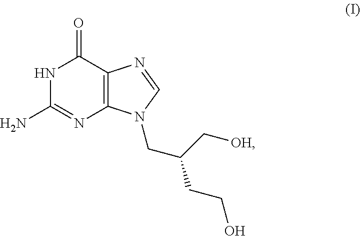 [SMILES: Nc1nc2c(ncn2C[C@H](CO)CCO)c(=O)[nH]1]
or a derivative or analog thereof, for treatment or prevention of the viral infection.

2. The method of claim 1 wherein the viral infection is selected from the group consisting of a viral infection caused by: (1) human herpesvirus 1 (HHV-1 or HSV-1), a member of the α-herpesvirus subfamily; (2) human herpesvirus 2 (HHV-2 or HSV-2), a member of the α-herpesvirus subfamily; (3) human herpresvirus 3 (HHV-3 or VZV (varicella zoster virus)), a member of the α-herpesvirus subfamily; (4) human herpesvirus 4 (HHV-4 or Epstein-Barr virus), a member of the γ-herpesvirus subfamily; (5) human herpesvirus 5 (HHV-5 or CMV (cytomegalovirus), a member of the β-herpesvirus subfamily; (6) human herpesvirus 6a (HHV-6a), a member of the β-herpesvirus subfamily; (7) human herpesvirus 6b (HHV-6b), a member of the β-herpesvirus subfamily; (8) human herpesvirus 7 (HHV-7), a member of the β-herpesvirus subfamily; and (9) human herpesvirus 8 (HHV-8 or KSHV (Kaposi's sarcoma associated herpesvirus), a member of the γ-herpesvirus subfamily.

3. The method of claim 2 wherein the viral infection is a viral infection caused by human herpesvirus 1 or human herpesvirus 2 and is herpes simplex virus infection.

4. The method of claim 2 wherein the viral infection is a viral infection caused by human herpesvirus 3 (varicella zoster virus).

5. The method of claim 2 wherein the viral infection is a viral infection caused by human herpesvirus 4 (Epstein-Barr virus).

6. The method of claim 5 wherein the viral infection caused by human herpesvirus 4 (Epstein-Barr virus) is an infection of a patient in an intensive care unit (ICU) and wherein the method decreases the time the patient spends on a ventilator.

7. The method of claim 2 wherein the viral infection is a viral infection caused by human herpesvirus 5 (cytomegalovirus).

8. The method of claim 2 wherein the viral infection is a viral infection caused by human herpesvirus 8 (Kaposi's sarcoma associated herpesvirus).

9. The method of claim 1 wherein the viral infection is a viral infection caused by hepatitis C virus (HCV).

10. The method of claim 1 wherein the viral infection is a viral infection caused by human immunodeficiency virus (HIV).

11. The method of claim 1 wherein the viral infection is a viral infection caused by an animal herpesvirus.

12. The method of claim 1 wherein the compound of Formula (I) is administered as the compound itself.

13. The method of claim 1 wherein the compound of Formula (I) or the derivative or analog thereof is administered as a pharmaceutical composition, wherein the pharmaceutical composition comprises: (1) the compound of Formula (I) or the derivative or analog thereof; and (2) at least one pharmaceutically acceptable carrier.

14. The method of claim 1 wherein the method comprises administration of a therapeutically effective quantity of an additional antiviral agent.

15. The method of claim 14 wherein the viral infection is an infection by HHV-5 (cytomegalovirus) and the additional antiviral agent is letermovir or a derivative or analog of letermovir.

16. The method of claim 15 wherein the additional antiviral agent is letermovir.

17. The method of claim 15 wherein the additional antiviral agent is a derivative or analog of letermovir.

18. The method of claim 14 wherein the viral infection is an infection by a virus selected from the group consisting of:
(a) cytomegalovirus or Epstein-Barr virus, wherein the additional antiviral agent is valganciclovir;
(b) cytomegalovirus, wherein the additional antiviral agent is selected from the group consisting of ganciclovir, cidofovir, and foscarnet;
(c) Epstein-Barr virus, wherein the additional antiviral agent is selected from the group consisting of ganciclovir, acyclovir, valaciclovir, cidofovir, adefovir, foscarnet, and romidepsin;
(d) herpes simplex virus, wherein the additional antiviral agent is selected from the group consisting of acyclovir, valaciclovir, famciclovir, and penciclovir;
(e) human immunodeficiency virus, wherein the additional antiviral agent is selected from the group consisting of nucleoside reverse transcriptase inhibitors, non-nucleoside reverse transcriptase inhibitors, protease inhibitors, and fusion inhibitors; and
(f) hepatitis C virus, wherein the additional antiviral agent is selected from the group consisting of sofosbuvir, ribavirin, pegylated interferon-α-2a, pegylated interferon-α-2b, boceprevir, telaprevir, ledipasvir, and simiprevir.

19. The method of claim 14 wherein the viral infection is an infection by Epstein-Barr virus and the additional therapeutic agent is a lytic induction agent selected from the group consisting of 5-fluorouracil, cisplatin, taxol, 5-iodo-2′-deoxyuridine, phorbol ester tetradecanoyl phorbol acetate, doxorubicin, gemcitabine, butyrate salts phenylbutyrate, arsenic trioxide, calcium ionophores, 5-azacytidine, 5-aza-2′-deoxycytidine, procaine, trichostatin A, trapoxin B, histone acetylating agents, histone deacetylase inhibitors, dexamethasone, rituximab, depsipeptides, vorinostat, romidepsin, belinostat, suberoylanilide hydroxamic acid, cinnamic acid hydroxamate, panobinostat, entinostat, mocetinostat, abexinostat, pracinostat, resminostat, givinostat, quisinostat, 7-(4-(3-ethynylphenylamino)-7-methoxyquinazolin-6-yloxy)-N-hydroxyheptanamide (CUDC-101), N-hydroxy-4-[[(2S)-3-methyl-2-phenylbutanoyl]amino]benzamide (AR-42), tefinostat, 2-(6-{[(6-fluoroquinolin-2-yl)methyl]amino}bicyclo[3.1.0]hex-3-yl)-N-hydroxypyrimidine-5-carboxamide (CHR-3996), 4SC-202, (E)-N1-(3-(dimethylamino)propyl)-N8-hydroxy-2-((naphthalen-1-yloxy)methyl)oct-2-enediamide (CG200745), rocinilinostat, 4,4′-(7-hydroxy-8-methylchroman-3,4-diyl)diphenol (ME-344), sulforaphane, kevetrin, and valproic acid.

20. The method of claim 14 wherein the additional antiviral agent is at least one compound selected from the group consisting of valomaciclovir stearate, octadecyloxyethyl-cidofovir, hexadecyloxypropyl-cidofovir, adefovir, amantadine, arbidol, brivudine, darunavir, docosanol, edoxudine, entecavir, fomivirsen, fosfonet, ibacitabine, immunovir, idoxuridine, imiquimod, inosine, loviride, raltegravir, maraviroc, moroxydine, nelfinavir, nexavir, oseltamivir, peramivir, pleconaril, podophyllotoxin, rimantidine, tenofovir, tipranavir, trifluridine, tromantidine, vicriviroc, vidarabine, viramidine, zanamivir, (2-amino-7-[(1,3-dihydroxy-2-propoxy)methyl]purine], (1'S,2′R)-9-[[1′,2′-bis(hydroxymethyl)cycloprop-1′-yl]methyl]guanine (A-5021), cyclopropavir, 2,4-diamino-6-R-[3-hydroxy-2(phosphonomethoxy)propoxy]-pyrimidine, (S)-9-(3-hydroxy-2-phosphonylmethoxypropyl)adenine (S-HPMPA), 3-deaza-9-(3-hydroxy-2-phosphonylmethoxypropyl)adenine (3-deaza-HPMPA), N-(4-chlorobenzyl)-1-methyl-6-(4-morpholinylmethyl)-4-oxo-1,4-dihydro-3-quinolinecarboxamine (PNU-183792), 2-bromo-5,6-dichloro-1-(β-D-ribofuranosyl)benzimidazole (BDCRB), maribavir, 3-hydroxy-2,2-dimethyl-N-[4-{[(5-dimethylamino)-1-naphthyl]-sulfonyl]-amino)phenyl}propamide (BAY 38-4766), N—[N-[4-(2-Aminothiazol-4-yl)phenyl]carbamoylmethyl]-N-[1(S)-phenylethyl]pyridine-4-carboxamide (BILS179BS), N-[5-(aminosulfonyl)-4-methyl-1,3-thiazol-2-yl]-N-methyl-2-{4-(2-pyridinyl)phenyl}acetamide (BAY 57-1293), 2H-3-(4-chlorophenyl)-3,4-dihydro-1,4-benzo-thiazine-2-carbonitrile 1,1-dioxide, and 2-chloro-3-pyridin-3-yl-5,6,7,8-tetrahydronindolizine-1-carboxamide (CMV423).

21. The method of claim 14 wherein the additional antiviral agent is selected from the group consisting of:
(a) an integrase inhibitor selected from the group consisting of elvitegravir, dolutegravir, raltegravir, and (6S)-2-(3-chloro-4-fluorobenzyl)-8-ethyl-10-hydroxy-N,6-dimethyl-1,9-dioxo-1,2,6,7,8,9-hexahydropyrazino[1′,2′:1,5]pyrrolo[2,3-d]pyridazine-4-carboxamide (MK-2048);
(b) an entry inhibitor selected from the group consisting of fostemsavir (BMS-663068), aplaviroc, (5,5′-((1E,1′E)-(methylenedisulfonyl)bis(ethane-2,1-diyl))bis(benzene-1,2,3-triol) (DCM205), and VIR-576;
(c) an interferon selected from the group consisting of interferon type I, an interferon type II, an interferon type III, and a pegylated interferon; and
(d) a syngergistic enhancer selected from the group consisting of hydroxyurea, leflunomide, mycophenolic acid, and resveratrol.

22. The method of claim 1 wherein the method comprises the step of administering a therapeutically effective quantity of a derivative or analog of H2G.

23. The method of claim 22 wherein the derivative or analog of H2G is selected from the group consisting of:
(a) a monophosphate derivative of H2G, a diphosphate derivative of H2G, and a triphosphate derivative of H2G;
(b) a phosphate prodrug analog of H2G;
(c) an analog of H2G selected from the group consisting of a compound of Formula (II) 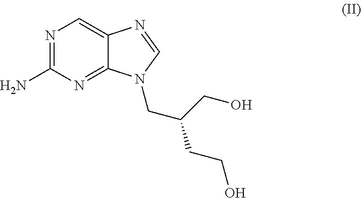 [SMILES: Nc1ncc2ncn(C[C@H](CO)CCO)c2n1]
and monophosphate derivatives, diphosphate derivatives, or triphosphate derivatives of analogs of Formula (II);
(d) an analog of H2G selected from the group consisting of a compound of Formula (III) 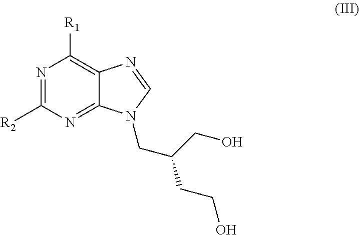
wherein:
(1) R₁ is hydrogen, hydroxy, mercapto, or amino; and
(2) R₂ is hydrogen, hydroxy, fluoro, chloro, or amino;
and monophosphate derivatives, diphosphate derivatives, or triphosphate derivatives of analogs of Formula (III);
(e) an analog of H2G selected from the group consisting of a compound of Formula (IV) 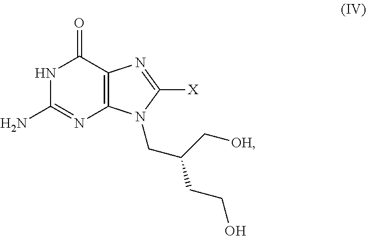
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (IV);
(f) an analog of H2G selected from the group consisting of a compound of Formula (V) 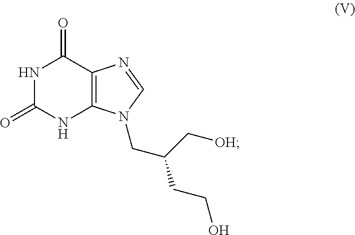 [SMILES: O=c1[nH]c(=O)c2ncn(C[C@H](CO)CCO)c2[nH]1]
and monophosphate derivatives, diphosphate derivatives, or triphosphate derivatives of analogs of Formula (V);
(g) an analog of H2G selected from the group consisting of a compound of Formula (VI) 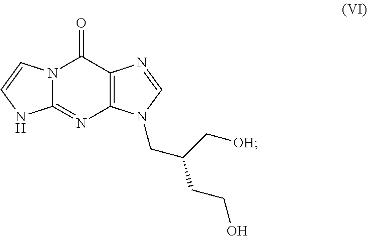 [SMILES: O=c1c2ncn(C[C@H](CO)CCO)c2nc2[nH]ccn12]
and monophosphate derivatives, diphosphate derivatives, or triphosphate derivatives of analogs of Formula (VI);
(h) an analog of H2G selected from the group consisting of a compound of Formula (VII) 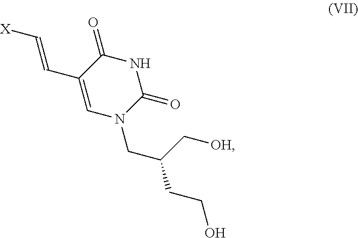
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (VII);
(i) an analog of H2G selected from the group consisting of a compound of Formula (VIII) 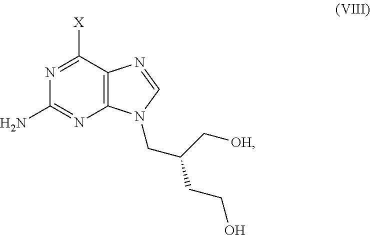
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (VIII);
(j) an analog of H2G selected from the group consisting of a compound of Formula (IX) 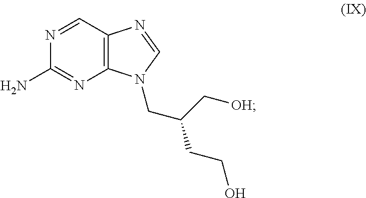 [SMILES: Nc1ncc2ncn(C[C@H](CO)CCO)c2n1]
and monophosphate derivatives, diphosphate derivatives, or triphosphate derivatives of analogs of Formula (IX);
(k) an analog of H2G selected from the group consisting of a compound of Formula (X) 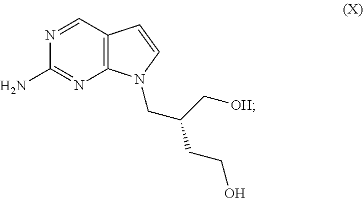 [SMILES: Nc1ncc2ccn(C[C@H](CO)CCO)c2n1]
and monophosphate derivatives, diphosphate derivatives, or triphosphate derivatives of analogs of Formula (X);
(l) an analog of H2G selected from the group consisting of a compound of Formula (XI) 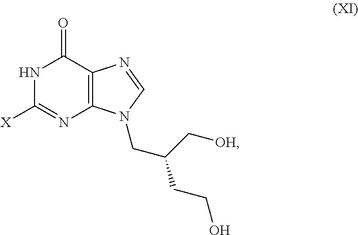
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (XI);
(m) an analog of H2G selected from the group consisting of a compound of Formula (XII) 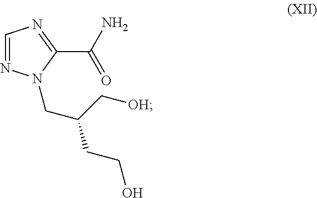 [SMILES: NC(=O)c1ncnn1C[C@H](CO)CCO]
and monophosphate derivatives, diphosphate derivatives, or triphosphate derivatives of analogs of Formula (XII);
(n) an analog of H2G selected from the group consisting of a compound of Formula (XIII) 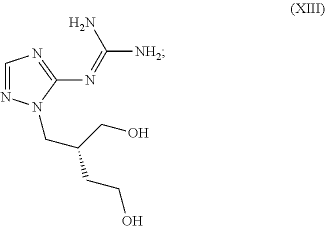 [SMILES: NC(N)=Nc1ncnn1C[C@H](CO)CCO]
and monophosphate derivatives, diphosphate derivatives, or triphosphate derivatives of analogs of Formula (XIII);
(o) an analog of H2G selected from the group consisting of a compound of Formula (XIV) 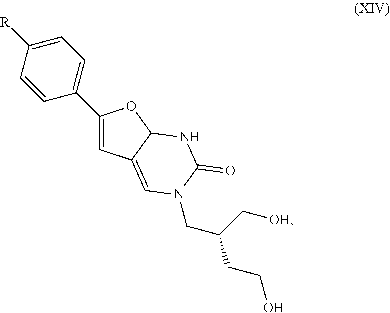
wherein R is selected from the group consisting of —(CH₂)ₙ—CH₃ wherein n is an integer from 0 to 11 and -(Phenyl)-p-(CH₂)ₙ—CH₃ wherein n is an integer from 1 to 10;
and monophosphate derivatives, diphosphate derivatives, or triphosphate derivatives of analogs of Formula (XIV);
(p) an analog of H2G selected from the group consisting of a compound of Formula (XV) 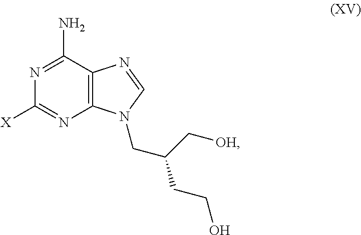
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (XV);
(q) an analog of H2G selected from the group consisting of a compound of Formula (XVI) 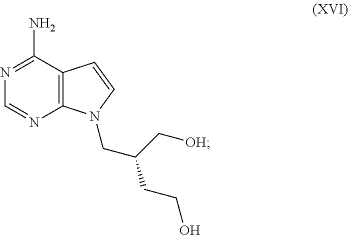 [SMILES: Nc1ncnc2c1ccn2C[C@H](CO)CCO]
and monophosphate derivatives, diphosphate derivatives, or triphosphate derivatives of analogs of Formula (XVI);
(r) an analog of H2G selected from the group consisting of a compound of Formula (XVII) 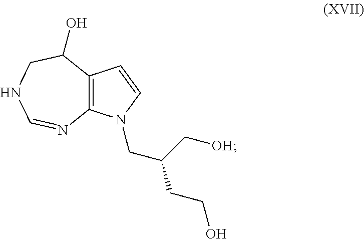 [SMILES: OCC[C@@H](CO)Cn1ccc2c1N=CNCC2O]
and monophosphate derivatives, diphosphate derivatives, or triphosphate derivatives of analogs of Formula (XVII);
(s) an ether or ester of a compound of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), or Formula (XVII); and
(t) an alkyl or arylalkyl derivative of a primary hydroxyl group of a compound of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), or Formula (XVII).

24. A pharmaceutical composition comprising:
(a) a therapeutically effective quantity of 9-(4-hydroxy-2-(hydroxymethyl)butyl)guanine (“H2G”) having the structure of Formula (I) 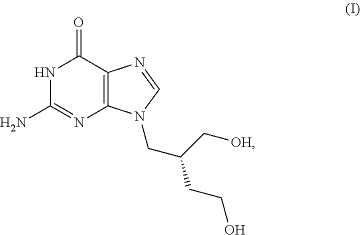 [SMILES: Nc1nc2c(ncn2C[C@H](CO)CCO)c(=O)[nH]1]
or a derivative or analog thereof; and
(b) a pharmaceutically acceptable carrier.

25. The pharmaceutical composition of claim 24 wherein the H2G or the derivative or analog of H2G is H2G.

26. The pharmaceutical composition of claim 24 wherein H2G or the derivative or analog of H2G is a derivative or analog of H2G.

27. The pharmaceutical composition of claim 26 wherein the derivative or analog of H2G is selected from the group consisting of:
(a) a monophosphate derivative of H2G, a diphosphate derivative of H2G, and a triphosphate derivative of H2G;
(b) a phosphate prodrug analog of H2G;
(c) an analog of H2G selected from the group consisting of a compound of Formula (II) 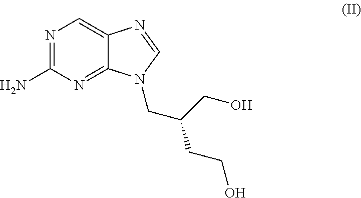 [SMILES: Nc1ncc2ncn(C[C@H](CO)CCO)c2n1]
and monophosphate derivatives, diphosphate derivatives, or triphosphate derivatives of analogs of Formula (II);
(d) an analog of H2G selected from the group consisting of a compound of Formula (III) 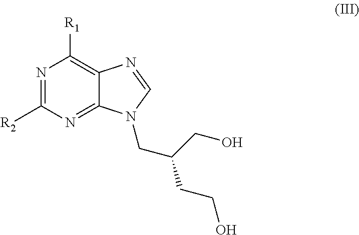
wherein:
(1) R₁ is hydrogen, hydroxy, mercapto, or amino; and
(2) R₂ is hydrogen, hydroxy, fluoro, chloro, or amino;
and monophosphate derivatives, diphosphate derivatives, or triphosphate derivatives of analogs of Formula (III);
(e) an analog of H2G selected from the group consisting of a compound of Formula (IV) 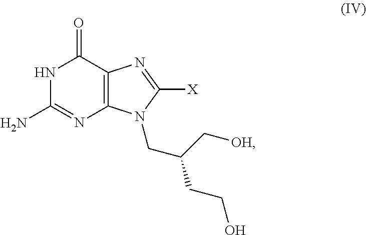
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (IV);
(f) an analog of H2G selected from the group consisting of a compound of Formula (V) 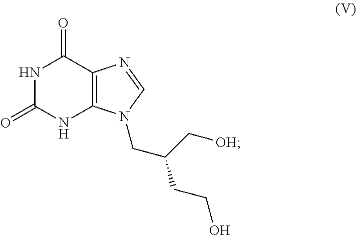 [SMILES: O=c1[nH]c(=O)c2ncn(C[C@H](CO)CCO)c2[nH]1]
and monophosphate derivatives, diphosphate derivatives, or triphosphate derivatives of analogs of Formula (V);
(g) an analog of H2G selected from the group consisting of a compound of Formula (VI) 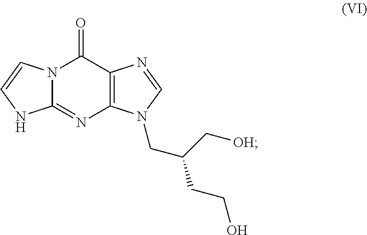 [SMILES: O=c1c2ncn(C[C@H](CO)CCO)c2nc2[nH]ccn12]
and monophosphate derivatives, diphosphate derivatives, or triphosphate derivatives of analogs of Formula (VI);
(h) an analog of H2G selected from the group consisting of a compound of Formula (VII) 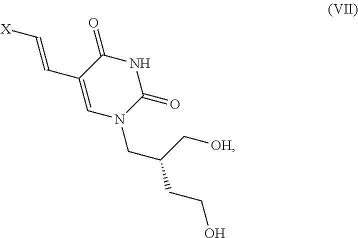
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (VII);
(i) an analog of H2G selected from the group consisting of a compound of Formula (VIII) 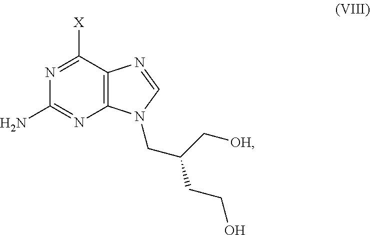
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (VIII);
(j) an analog of H2G selected from the group consisting of a compound of Formula (IX) 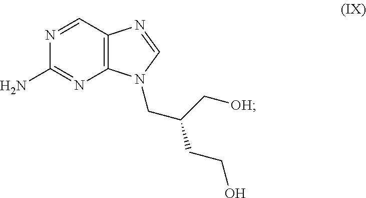 [SMILES: Nc1ncc2ncn(C[C@H](CO)CCO)c2n1]
and monophosphate derivatives, diphosphate derivatives, or triphosphate derivatives of analogs of Formula (IX);
(k) an analog of H2G selected from the group consisting of a compound of Formula (X) 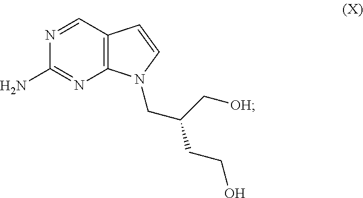 [SMILES: Nc1ncc2ccn(C[C@H](CO)CCO)c2n1]
and monophosphate derivatives, diphosphate derivatives, or triphosphate derivatives of analogs of Formula (X);
(l) an analog of H2G selected from the group consisting of a compound of Formula (XI) 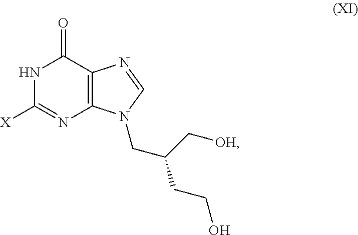
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (XI);
(m) an analog of H2G selected from the group consisting of a compound of Formula (XII) 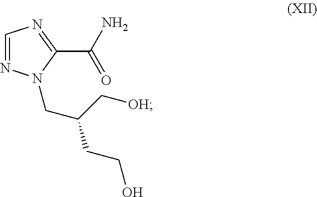 [SMILES: NC(=O)c1ncnn1C[C@H](CO)CCO]
and monophosphate derivatives, diphosphate derivatives, or triphosphate derivatives of analogs of Formula (XII);
(n) an analog of H2G selected from the group consisting of a compound of Formula (XIII) 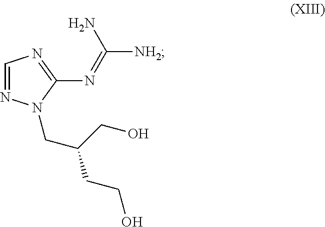 [SMILES: NC(N)=Nc1ncnn1C[C@H](CO)CCO]
and monophosphate derivatives, diphosphate derivatives, or triphosphate derivatives of analogs of Formula (XIII);
(o) an analog of H2G selected from the group consisting of a compound of Formula (XIV) 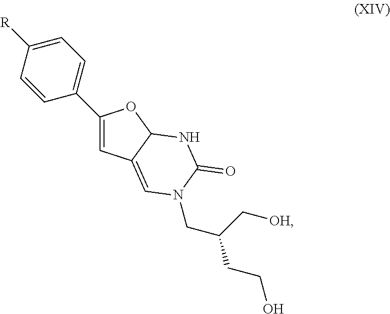
wherein R is selected from the group consisting of —(CH₂)ₙ—CH₃ wherein n is an integer from 0 to 11 and -(Phenyl)-p-(CH₂)ₙ—CH₃ wherein n is an integer from 1 to 10;
and monophosphate derivatives, diphosphate derivatives, or triphosphate derivatives of analogs of Formula (XIV);
(p) an analog of H2G selected from the group consisting of a compound of Formula (XV) 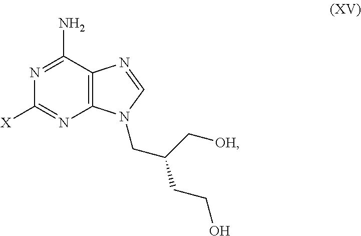
wherein X is selected from the group consisting of fluoro, chloro, bromo, iodo, —O-alkyl, and —S-alkyl, wherein the alkyl moieties are optionally substituted;
and monophosphate derivatives, diphosphate derivatives, or triphosphate derivatives of analogs of Formula (XV);
(q) an analog of H2G selected from the group consisting of a compound of Formula (XVI) 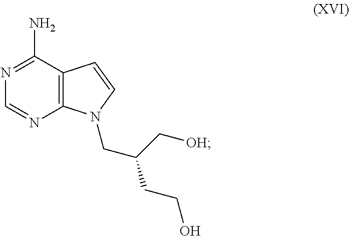 [SMILES: Nc1ncnc2c1ccn2C[C@H](CO)CCO]
and monophosphate derivatives, diphosphate derivatives, or triphosphate derivatives of analogs of Formula (XVI);
(r) an analog of H2G selected from the group consisting of a compound of Formula (XVII) 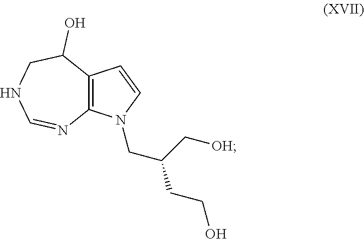 [SMILES: OCC[C@@H](CO)Cn1ccc2c1N=CNCC2O]
and monophosphate derivatives, diphosphate derivatives, or triphosphate derivatives of analogs of Formula (XVII);
(s) an ether or ester of a compound of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), or Formula (XVII); and
(t) an alkyl or arylalkyl derivative of a primary hydroxyl group of a compound of Formula (I), Formula (II), Formula (III), Formula (IV), Formula (V), Formula (VI), Formula (VII), Formula (VIII), Formula (IX), Formula (X), Formula (XI), Formula (XII), Formula (XIII), Formula (XIV), Formula (XV), Formula (XVI), or Formula (XVII).

28. The pharmaceutical composition of claim 24 wherein the pharmaceutical composition comprises a therapeutically effective quantity of an additional antiviral agent.

29. The pharmaceutical composition of claim 28 wherein the additional antiviral agent is letermovir or a derivative or analog of letermovir.

30. The pharmaceutical composition of claim 28 wherein the additional antiviral agent is selected from the group consisting of: valganciclovir; ganciclovir; cidofovir; foscarnet; acyclovir; valaciclovir; adefovir; romidepsin; famciclovir; penciclovir; nucleoside reverse transcriptase inhibitors for inhibition of replication of human immunodeficiency virus; non-nucleoside reverse transcriptase inhibitors for inhibition of replication of human immunodeficiency virus; protease inhibitors for inhibition of replication of human immunodeficiency virus; fusion inhibitors for inhibition of replication of human immunodeficiency virus; sofosbuvir; ribavirin; pegylated interferon-α-2a; pegylated interferon-α-2b; boceprevir; telaprevir; ledipasvir; and simiprevir; a lytic induction agent for induction of the lytic cycle of Epstein-Barr virus selected from the group consisting of 5-fluorouracil, cisplatin, taxol, 5-iodo-2′-deoxyuridine, phorbol ester tetradecanoyl phorbol acetate, doxorubicin, gemcitabine, butyrate salts phenylbutyrate, arsenic trioxide, calcium ionophores, 5-azacytidine, 5-aza-2′-deoxycytidine, procaine, trichostatin A, trapoxin B, histone acetylating agents, histone deacetylase inhibitors, dexamethasone, rituximab, depsipeptides, vorinostat, romidepsin, belinostat, suberoylanilide hydroxamic acid, cinnamic acid hydroxamate, panobinostat, entinostat, mocetinostat, abexinostat, pracinostat, resminostat, givinostat, quisinostat, 7-(4-(3-ethynylphenylamino)-7-methoxyquinazolin-6-yloxy)-N-hydroxyheptanamide (CUDC-101), N-hydroxy-4-[[(2S)-3-methyl-2-phenylbutanoyl]amino]benzamide (AR-42), tefinostat, 2-(6-{[(6-fluoroquinolin-2-yl)methyl]amino}bicyclo[3.1.0]hex-3-yl)-N-hydroxypyrimidine-5-carboxamide (CHR-3996), 4SC-202, (E)-N1-(3-(dimethylamino)propyl)-N8-hydroxy-2-((naphthalen-1-yloxy)methyl)oct-2-enediamide (CG200745), rocinilinostat, 4,4′-(7-hydroxy-8-methylchroman-3,4-diyl)diphenol (ME-344), sulforaphane, kevetrin, and valproic acid; an antiviral agent selected from the group consisting of valomaciclovir stearate, octadecyloxyethyl-cidofovir, hexadecyloxypropyl-cidofovir, adefovir, amantadine, arbidol, brivudine, darunavir, docosanol, edoxudine, entecavir, fomivirsen, fosfonet, ibacitabine, immunovir, idoxuridine, imiquimod, inosine, loviride, raltegravir, maraviroc, moroxydine, nelfinavir, nexavir, oseltamivir, peramivir, pleconaril, podophyllotoxin, rimantidine, tenofovir, tipranavir, trifluridine, tromantidine, vicriviroc, vidarabine, viramidine, zanamivir, (2-amino-7-[(1,3-dihydroxy-2-propoxy)methyl]purine], (1'S,2′R)-9-[[1′,2′-bis(hydroxymethyl)cycloprop-1′-yl]methyl]guanine (A-5021), cyclopropavir, 2,4-diamino-6-R-[3-hydroxy-2(phosphonomethoxy)propoxy]-pyrimidine, (S)-9-(3-hydroxy-2-phosphonylmethoxypropyl)adenine (S-HPMPA), 3-deaza-9-(3-hydroxy-2-phosphonylmethoxypropyl)adenine (3-deaza-HPMPA), N-(4-chlorobenzyl)-1-methyl-6-(4-morpholinylmethyl)-4-oxo-1,4-dihydro-3-quinolinecarboxamine (PNU-183792), 2-bromo-5,6-dichloro-1-(β-D-ribofuranosyl)benzimidazole (BDCRB), maribavir, 3-hydroxy-2,2-dimethyl-N-[4-{[(5-dimethylamino)-1-naphthyl]-sulfonyl]-amino)phenyl}propamide (BAY 38-4766), N—[N-[4-(2-Aminothiazol-4-yl)phenyl]carbamoylmethyl]-N-[1(S)-phenylethyl]pyridine-4-carboxamide (BILS179BS), N-[5-(aminosulfonyl)-4-methyl-1,3-thiazol-2-yl]-N-methyl-2-{4-(2-pyridinyl)phenyl}acetamide (BAY 57-1293), 2H-3-(4-chlorophenyl)-3,4-dihydro-1,4-benzo-thiazine-2-carbonitrile 1,1-dioxide, and 2-chloro-3-pyridin-3-yl-5,6,7,8-tetrahydronindolizine-1-carboxamide (CMV423); an integrase inhibitor selected from the group consisting of elvitegravir, dolutegravir, raltegravir, and (6S)-2-(3-chloro-4-fluorobenzyl)-8-ethyl-10-hydroxy-N,6-dimethyl-1,9-dioxo-1,2,6,7,8,9-hexahydropyrazino[1′,2′:1,5]pyrrolo[2,3-d]pyridazine-4-carboxamide (MK-2048); an entry inhibitor selected from the group consisting of fostemsavir (BMS-663068), aplaviroc, (5,5′-((1E,1′E)-(methylenedisulfonyl)bis(ethane-2,1-diyl))bis(benzene-1,2,3-triol) (DCM205), and VIR-576; an interferon selected from the group consisting of an interferon type I, an interferon type II, an interferon type III, and a pegylated interferon;
and a synergistic enhancer selected from the group consisting of hydroxyurea, leflunomide, mycophenolic acid, and resveratrol.

31. The pharmaceutical composition of claim 24 wherein the pharmaceutical composition is formulated for oral administration and is in a dosage form selected from the group consisting of solutions, suspensions, tablets, dispersible tablets, pills, capsules, troches, granules, bulk powders, sustained release formulations and elixirs.

32. The pharmaceutical composition of claim 24 wherein the pharmaceutical composition is formulated for parenteral administration and is in a dosage form selected from the group consisting of sterile solutions and suspensions.

33. The pharmaceutical composition of claim 24 wherein the pharmaceutical composition is formulated in a dosage form selected from the group consisting of:
(a) a dosage form formulated for administration via a transdermal patch;
(b) a dosage form formulated for administration via a dry powder inhaler;
(c) a dosage form formulated as an oil-water emulsion;
(d) a dosage form formulated as a unit-dosage form;
(e) a dosage form formulated as a multiple-dosage form;
(f) a dosage form formulated as a solid dosage form that includes at least one ingredient selected from the group consisting of: a binder; a lubricant; a diluent; a glidant; a disintegrating agent; a coloring agent; a sweetening agent; a flavoring agent; a wetting agent; an emetic coating; and a film coating;
(g) a dosage form formulated to include an enteric coating;
(h) a dosage form formulated as a sterile lyophilized powder;
(i) a dosage form formulated as a topical mixture;
(j) a dosage form formulated as an aerosol for topical application;
(k) a dosage form formulated for administration by inhalation through the mouth or nasal passages;
(l) a dosage form formulated for topical application to the skin or mucous membranes; and
(m) a dosage form formulated for rectal administration.

34. The pharmaceutical composition of claim 24 wherein the pharmaceutically acceptable carrier comprises a vehicle selected from the group consisting of water, saline, aqueous dextrose, glycerol, glycols, and ethanol.

35. The pharmaceutical composition of claim 24 wherein the pharmaceutically acceptable carrier comprises a nontoxic auxiliary substance selected from the group of wetting agents, emulsifying agents, solubilizing agents, and pH buffering agents.

36. The pharmaceutical composition of claim 32 wherein the pharmaceutical composition includes a vehicle selected from the group consisting of aqueous vehicles, nonaqueous vehicles, antimicrobial agents, isotonic agents, buffers, antioxidants, local anesthetics, suspending and dispersing agents, emulsifying agents, sequestering agents, and chelating agents.

37. The pharmaceutical composition of claim 24 wherein the pharmaceutical composition is formulated to target the therapeutically effective quantity of H2G or the derivative or analog thereof to a particular organ, tissue, receptor, or other area of the body of a subject to be treated.

38. The pharmaceutical composition of claim 32 wherein the pharmaceutical composition is formulated to target the therapeutically effective quantity of the additional antiviral agent to a particular organ, tissue, receptor, or other area of the body of a subject to be treated.

39. The pharmaceutical composition of claim 32 wherein the pharmaceutical composition is formulated to target both the therapeutically effective quantity of H2G or the derivative or analog thereof and the additional antiviral agent to a particular organ, tissue, receptor, or other area of the body of a subject to be treated.

40. An article of manufacture comprising:
(a) a therapeutically effective quantity of H2G or a derivative or analog of H2G of claim 1;
(b) packaging material to package the therapeutically effective quantity of H2G or a derivative or analog of H2G; and
(c) a label indicating that the H2G or the derivative or analog thereof is useful for treating a viral infection and providing instructions for its use.

41. An article of manufacture comprising:
(a) a therapeutically effective quantity of H2G or a derivative or analog of H2G of claim 1;
(b) an additional antiviral agent;
(c) packaging material to package the therapeutically effective quantity of H2G or a derivative or analog of H2G and the additional antiviral agent; and
(d) a label indicating that the H2G or the derivative or analog thereof and the additional antiviral agent are useful for treating a viral infection and providing instructions for its use.

42. A method for the treatment of a disease or condition selected from the group consisting of systemic sclerosis, myalgic encephalomyelitis/chronic fatigue syndrome, Alzheimer's disease, systemic lupus erythematosus, multiple sclerosis and Graves' disease comprising administering a therapeutically effective quantity of 9-(4-hydroxy-2-(hydroxymethyl)butyl)guanine (“H2G”) having the structure of Formula (I) 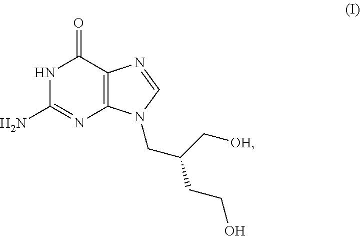 [SMILES: Nc1nc2c(ncn2C[C@H](CO)CCO)c(=O)[nH]1]
or a derivative or analog thereof for treatment of the disease or condition.

43. The method of claim 42 wherein the method comprises administering a therapeutically effective quantity of H2G.

44. The method of claim 42 wherein the compound of Formula (I) or the derivative or analog thereof is administered as a pharmaceutical composition, wherein the pharmaceutical composition comprises: (1) the compound of Formula (I) or the derivative or analog thereof; and (2) at least one pharmaceutically acceptable carrier.

45. The method of claim 42 wherein the method is for the treatment of a disease or condition selected from the group consisting of:
(a) systemic sclerosis and wherein the method further comprises the administration of a therapeutically effective quantity of an agent selected from the group consisting of non-steroidal anti-inflammatory drugs, steroids, calcium channel blockers, iloprost, bosentan, methotrexate, ciclosporin, penicillamine, angiotensin converting enzyme inhibitors, cyclophosphamide, epoprostenol, antithymocyte globulin, and mycophenolate mofetil;
(b) myalgic encephalomyelitis/chronic fatigue syndrome and wherein the method further comprises the administration of a therapeutically effective quantity of an agent selected from the group consisting of antidepressants and immune system stimulating agents;
(c) Alzheimer's disease and wherein the method further comprises the administration of a therapeutically effective quantity of an agent selected from the group consisting of tacrine, rivastigmine, galantamine, donezepil, memantine, and huperzine A;
(d) systemic lupus erythematosus and wherein the method further comprises the administration of a therapeutically effective quantity of an agent selected from the group consisting of non-steroidal anti-inflammatory drugs, prednisone, hydroxychloroquine, methotrexate, azathioprine, cyclophosphamide, mycophenolic acid, belimumab, atacicept, lupuzor, intravenous immunoglobulin, infliximab, anakinra, rituximab, tocilizumab, abatacept, and leflunomide;
(e) multiple sclerosis and wherein the method further comprises the administration of a therapeutically effective quantity of an agent selected from the group consisting of interferon-β1a, interferon-β1b, glatiramer acetate, mitoxantrone, natalizumab, fingolimod, and dimethyl fumarate;
(f) idiopathic pulmonary fibrosis and wherein the method further comprises the administration of a therapeutically effective quantity of an agent selected from the group consisting of pirfenidone, nitedanib, simtuzumab, tralokimab, lebrikizumab, and FG-3019; and
(g) Graves' disease and wherein the method further comprises the administration of a therapeutically effective quantity of an agent selected from the group consisting of carbimazole, methimazole, and propylthiouracil.

46. A method for the treatment of cancer comprising administering a therapeutically effective quantity of 9-(4-hydroxy-2-(hydroxymethyl)butyl)guanine (“H2G”) having the structure of Formula (I) 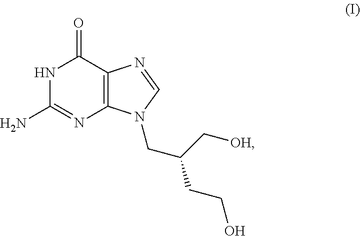 [SMILES: Nc1nc2c(ncn2C[C@H](CO)CCO)c(=O)[nH]1]
or a derivative or analog thereof for treatment of the cancer.

47. The method of claim 46 wherein the cancer is selected from the group consisting of gastric carcinoma, lymphoma, Hodgkin's disease, nasopharygeal carcinoma, breast cancer, lung cancer, colon cancer, and prostate cancer.

48. The method of claim 47 wherein the cancer is lymphoma and wherein the lymphoma is selected from the group consisting of B-cell lymphoma, an AIDS-related lymphoma, and Burkitt's lymphoma.

49. The method of claim 46 wherein the method comprises administering a therapeutically effective quantity of H2G.

50. The method of claim 46 wherein the compound of Formula (I) or the derivative or analog thereof is administered as a pharmaceutical composition, wherein the pharmaceutical composition comprises: (1) the compound of Formula (I) or the derivative or analog thereof; and (2) at least one pharmaceutically acceptable carrier.

51. The method of claim 46 wherein the method further comprises the administration of an additional anti-neoplastic agent.

52. The method of claim 51 wherein the additional anti-neoplastic agent is selected from the group consisting of mechlorethamine, cyclophosphamide, ifosfamide, melphalan, chlorambucil, hexamethylmelamine, thiotepa, busulfan, carmustine, streptozocin, dacarbazine, temozolomide, methotrexate, 5-fluorouracil, cytarabine, gemcitabine, 6-mercaptopurine, 6-thioguanine, pentostatin, vinblastine, vincristine, paclitaxel, docetaxel, topotecan, irinotecan, dactinomycin, daunorubicin, doxorubicin, bleomycin, mitomycin C, L-asparaginase, interferon-alfa, interleukin-2, cisplatin, carboplatin, mitoxantrone, hydroxyurea, N-methylhydrazine, mitotane, aminoglutethimide, imatinib, prednisone, prednisolone, methylprednisolone, dexamethasone, betamethasone, triamcinolone, hydroxyprogesterone, medroxyprogesterone, megestrol acetate, diethylstilbestrol, ethinyl estradiol, tamoxifen, anastrozole, testosterone propionate, fluoxymesterone, flutamine, leuprolide, trastuzumab, rituximab, alemtuzumab, bevacizumab, cetuximab, gemtuzumab, ibritumomab, panitumumab, tositumomab, and an interferon.
